SEQUENCE LISTING (41 sequences)

SEQ ID NO: 1 - DNA, 2309 bp, Artificial Sequence
  hAP4M1 opt vector
ctgcgcgctcgctcgctcactgaggccgcccgggcaaagcccgggcgtcgggcgacctttggtcgcccggcctcagtgagcgagcgagcgcgcagagagggagtggggttcggtacccgccggcggggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtcgcgcgcgctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagcgtcgacgccaccatgatctcccagttcttcatcctgtcatcgaagggcgaccctctgatctacaaggatttccgcggagactccggaggacgagatgtggcggaactcttctaccggaagctcactggcctgccgggagatgagtccccggtcgtcatgcaccatcacggccgccatttcatccatattcgccactctgggctgtacctggtggtcacaaccagcgaaaacgtcagcccgtttagcctcctggagttacttagccgcttggccactctcctgggagactattgtggctccctgggcgaaggaactatctccagaaacgtggccctggtgtacgaactcctcgacgaagtgctggactacggctacgtgcagaccacctcgaccgagatgctgcggaacttcattcagaccgaggctgtggtgtcgaagccttttagcctcttcgacctctcctctgtgggattgtttggtgccgaaactcagcagtccaaggtcgccccaagctcagccgcctcaagacctgtgctgagctcgagatcagatcagagccagaagaacgaggtgttcctggacgtggtggaacggcttagcgtcctgatcgcctccaacgggtcgctgctgaaggtcgacgtccagggagaaatccggctgaagtcattcctcccatcgggctcagaaatgcggatcggcctgactgaggagttctgcgtgggaaagagcgaactgagaggatacggccccggcattagagtggacgaagtctccttccattcctccgtgaacctggacgagttcgagtcccaccgcatcctgcggctccaaccgccacagggggaactgaccgtgatgcgctaccagctgtccgacgatctgccgagcccccttcctttccgcttgttcccgtccgtgcaatgggataggggctccggaaggctccaagtgtaccttaagctgcgctgcgatctgctctcgaaaagccaggcgctgaacgtgcgcctgcaccttcctctgccgaggggagtggtgtccctgtcccaagagctgtcctcgcccgaacagaaggccgagctggcagagggtgctctccgctgggacctacccagagtgcagggaggctcacagctgagcggactgttccagatggacgtgcccggacctcctggacccccatcccacggtctgtccacgtccgcctcacctctgggtctggggcctgcaagcttgagcttcgaacttccgcggcatacttgctccggtctgcaagtccggttcctccggctggcgttcaggccgtgcggcaatgccaacccgcacaaatgggtccggcacctgtcgcactccgacgcttacgtgattcggatttgataagcgatcgccagaacctcgaggaccacggcgcgcctttgcggatccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagctttggacgcgtaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttc

SEQ ID NO: 2 - DNA, 106 bp, Artificial Sequence
  5′ ITR
ctgcgcgctcgctcgctcactgaggccgcccgggcaaagcccgggcgtcgggcgacctttggtcgcccggcctcagtgagcgagcgagcgcgcagagagggagtgg

SEQ ID NO: 3 - DNA, 164 bp, Artificial Sequence
  JeT promotor
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 4 - DNA, 156 bp, Artificial Sequence
  Intron
ctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacag

SEQ ID NO: 5 - DNA, 1365 bp, Artificial Sequence
  hAP4M1opt
atgatctcccagttcttcatcctgtcatcgaagggcgaccctctgatctacaaggatttccgcggagactccggaggacgagatgtggcggaactcttctaccggaagctcactggcctgccgggagatgagtccccggtcgtcatgcaccatcacggccgccatttcatccatattcgccactctgggctgtacctggtggtcacaaccagcgaaaacgtcagcccgtttagcctcctggagttacttagccgcttggccactctcctgggagactattgtggctccctgggcgaaggaactatctccagaaacgtggccctggtgtacgaactcctcgacgaagtgctggactacggctacgtgcagaccacctcgaccgagatgctgcggaacttcattcagaccgaggctgtggtgtcgaagccttttagcctcttcgacctctcctctgtgggattgtttggtgccgaaactcagcagtccaaggtcgccccaagctcagccgcctcaagacctgtgctgagctcgagatcagatcagagccagaagaacgaggtgttcctggacgtggtggaacggcttagcgtcctgatcgcctccaacgggtcgctgctgaaggtcgacgtccagggagaaatccggctgaagtcattcctcccatcgggctcagaaatgcggatcggcctgactgaggagttctgcgtgggaaagagcgaactgagaggatacggccccggcattagagtggacgaagtctccttccattcctccgtgaacctggacgagttcgagtcccaccgcatcctgcggctccaaccgccacagggggaactgaccgtgatgcgctaccagctgtccgacgatctgccgagcccccttcctttccgcttgttcccgtccgtgcaatgggataggggctccggaaggctccaagtgtaccttaagctgcgctgcgatctgctctcgaaaagccaggcgctgaacgtgcgcctgcaccttcctctgccgaggggagtggtgtccctgtcccaagagctgtcctcgcccgaacagaaggccgagctggcagagggtgctctccgctgggacctacccagagtgcagggaggctcacagctgagcggactgttccagatggacgtgcccggacctcctggacccccatcccacggtctgtccacgtccgcctcacctctgggtctggggcctgcaagcttgagcttcgaacttccgcggcatacttgctccggtctgcaagtccggttcctccggctggcgttcaggccgtgcggcaatgccaacccgcacaaatgggtccggcacctgtcgcactccgacgcttacgtgattcggatttgataa

SEQ ID NO: 6 - DNA, 254 bp, Artificial Sequence
  BGHpA
ctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagct

SEQ ID NO: 7 - DNA, 169 bp, Artificial Sequence
  3′-ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttc

SEQ ID NO: 8 - DNA, 20 bp, Artificial Sequence
  FGEN-AP4M1 WT
tagaagccagccatagtggt

SEQ ID NO: 9 - DNA, 20 bp, Artificial Sequence
  RGEN-AP4M1 WT
gagcaagtgtgtggacctga

SEQ ID NO: 10 - DNA, 20 bp, Artificial Sequence
  FGEN_AP4M1 mut
cggtcgctaccattaccagt

SEQ ID NO: 11 - DNA, 20 bp, Artificial Sequence
  RGEN-AP4M1 mut
tgtttcagtgtcccatctgc

SEQ ID NO: 12 - DNA, 106 bp, Artificial Sequence; identical to SEQ ID NO: 2
  Nucleic acid sequence of a mutant AAV2 ITR

SEQ ID NO: 13 - DNA, 169 bp, Artificial Sequence; identical to SEQ ID NO: 7
  Nucleic acid sequence of a wildtype AAV2 ITR

SEQ ID NO: 14 - DNA, 130 bp, Artificial Sequence
  Nucleic acid sequence of a 5′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 15 - DNA, 130 bp, Artificial Sequence
  Nucleic acid sequence of a 3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 168 bp, Artificial Sequence
  Nucleic acid sequence of a 5′ ITR
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatct

SEQ ID NO: 17 - DNA, 168 bp, Artificial Sequence
  Nucleic acid sequence of a 5′ ITR
agatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccc

SEQ ID NO: 18 - DNA, 170 bp, Artificial Sequence
  Nucleic acid sequence of a 3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttc

SEQ ID NO: 19 - DNA, 104 bp, Artificial Sequence
  Nucleic acid sequence of a 5′ ITR
tgcgcgctcgctcgctcactgaggccgcccgggcaaagcccgggcgtcgggcgacctttggtcgcccggcctcagtgagcgagcgagcgcgcagagagggagtg

SEQ ID NO: 20 - DNA, 106 bp, Artificial Sequence
  Nucleic acid sequence of a 3′ ITR
ccactccctctctgcgcgctcgctcgctcactgaggccgggcgaccaaaggtcgcccgacgcccgggctttgcccgggcggcctcagtgagcgagcgagcgcgcca

SEQ ID NO: 21 - DNA, 225 bp, Artificial Sequence
  Nucleic acid sequence of an MeP229 promoter
caattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 22 - DNA, 241 bp, Artificial Sequence
  Nucleic acid sequence of a U6 promoter
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 23 - DNA, 448 bp, Artificial Sequence
  Nucleic acid sequence of a Synapsin promoter 1
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 24 - DNA, 469 bp, Artificial Sequence
  Nucleic acid sequence of a Synapsin promoter 2
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 25 - DNA, 302 bp, Artificial Sequence
  Nucleic acid sequence of a CBA promoter
acgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgg

SEQ ID NO: 26 - DNA, 332 bp, Artificial Sequence
  Nucleic acid sequence of a CBh promoter
ggtaccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacag

SEQ ID NO: 27 - DNA, 449 bp, Artificial Sequence
  Nucleic acid sequence of a hSyn promoter
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagt

SEQ ID NO: 28 - DNA, 223 bp, Artificial Sequence
  Nucleic acid sequence of a MeCP2 promoter
caattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccgga

SEQ ID NO: 29 - DNA, 332 bp, Artificial Sequence; identical to SEQ ID NO: 26
  Nucleic acid sequence of a JeT+I promoter

SEQ ID NO: 30 - DNA, 257 bp, Artificial Sequence
  Nucleic acid sequence of a CMV sequence
tacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatct

SEQ ID NO: 31 - DNA, 93 bp, Artificial Sequence
  Nucleic acid sequence of a CBA Exon 1
ggagtcgctgcgcgctgccttcgccccgtgccccgctccgccgccgcctcgcgccgcccgccccggctctgactgaccgcgttactcccacag

SEQ ID NO: 32 - DNA, 43 bp, Artificial Sequence
  Nucleic acid sequence of a CBA Intron 1
gtgagcgggcgggacggcccttctcctccgggctgtaattagc

SEQ ID NO: 33 - DNA, 92 bp, Artificial Sequence
  Nucleic acid sequence of an MVM Intron
aagaggtaagggtttaagggatggttggttggtggggtattaatgtttaattacctggagcacctgcctgaaatcactttttttcaggttgg

SEQ ID NO: 34 - DNA, 62 bp, Artificial Sequence
  Nucleic acid sequence of SpA
gcggccgcctcgagaataaagagctcagatgcatcgatcagagtgtgttggttttttgtgtg

SEQ ID NO: 35 - DNA, 10 bp, Artificial Sequence
  Nucleic acid sequence of a Kozak sequence
gccaccatgg

SEQ ID NO: 36 - DNA, 586 bp, Artificial Sequence
  Nucleic acid sequence of a WPRE
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccg

SEQ ID NO: 37 - DNA, 816 bp, Artificial Sequence
  Nucleic acid sequence of a kanamycin resistance gene
atgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 38 - DNA, 29 bp, Artificial Sequence
  Nucleic acid sequence of a resistance gene promoter
ttcaaatatgtatccgctcatgagacaat

SEQ ID NO: 39 - DNA, 328 bp, Artificial Sequence
  Nucleic acid sequence of a UsP promoter
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtcgcgcgcgctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacag

SEQ ID NO: 40 - DNA, 1362 bp, Artificial Sequence
  hAP4M1opt
atgatctcccagttcttcatcctgtcatcgaagggcgaccctctgatctacaaggatttccgcggagactccggaggacgagatgtggcggaactcttctaccggaagctcactggcctgccgggagatgagtccccggtcgtcatgcaccatcacggccgccatttcatccatattcgccactctgggctgtacctggtggtcacaaccagcgaaaacgtcagcccgtttagcctcctggagttacttagccgcttggccactctcctgggagactattgtggctccctgggcgaaggaactatctccagaaacgtggccctggtgtacgaactcctcgacgaagtgctggactacggctacgtgcagaccacctcgaccgagatgctgcggaacttcattcagaccgaggctgtggtgtcgaagccttttagcctcttcgacctctcctctgtgggattgtttggtgccgaaactcagcagtccaaggtcgccccaagctcagccgcctcaagacctgtgctgagctcgagatcagatcagagccagaagaacgaggtgttcctggacgtggtggaacggcttagcgtcctgatcgcctccaacgggtcgctgctgaaggtcgacgtccagggagaaatccggctgaagtcattcctcccatcgggctcagaaatgcggatcggcctgactgaggagttctgcgtgggaaagagcgaactgagaggatacggccccggcattagagtggacgaagtctccttccattcctccgtgaacctggacgagttcgagtcccaccgcatcctgcggctccaaccgccacagggggaactgaccgtgatgcgctaccagctgtccgacgatctgccgagcccccttcctttccgcttgttcccgtccgtgcaatgggataggggctccggaaggctccaagtgtaccttaagctgcgctgcgatctgctctcgaaaagccaggcgctgaacgtgcgcctgcaccttcctctgccgaggggagtggtgtccctgtcccaagagctgtcctcgcccgaacagaaggccgagctggcagagggtgctctccgctgggacctacccagagtgcagggaggctcacagctgagcggactgttccagatggacgtgcccggacctcctggacccccatcccacggtctgtccacgtccgcctcacctctgggtctggggcctgcaagcttgagcttcgaacttccgcggcatacttgctccggtctgcaagtccggttcctccggctggcgttcaggccgtgcggcaatgccaacccgcacaaatgggtccggcacctgtcgcactccgacgcttacgtgattcggatttga

SEQ ID NO: 41 - DNA, 5819 bp, Artificial Sequence
  hAP4M1 opt vector
ctgcgcgctcgctcgctcactgaggccgcccgggcaaagcccgggcgtcgggcgacctttggtcgcccggcctcagtgagcgagcgagcgcgcagagagggagtggggttcggtacccgccggcggggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtcgcgcgcgctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagcgtcgacgccaccatgatctcccagttcttcatcctgtcatcgaagggcgaccctctgatctacaaggatttccgcggagactccggaggacgagatgtggcggaactcttctaccggaagctcactggcctgccgggagatgagtccccggtcgtcatgcaccatcacggccgccatttcatccatattcgccactctgggctgtacctggtggtcacaaccagcgaaaacgtcagcccgtttagcctcctggagttacttagccgcttggccactctcctgggagactattgtggctccctgggcgaaggaactatctccagaaacgtggccctggtgtacgaactcctcgacgaagtgctggactacggctacgtgcagaccacctcgaccgagatgctgcggaacttcattcagaccgaggctgtggtgtcgaagccttttagcctcttcgacctctcctctgtgggattgtttggtgccgaaactcagcagtccaaggtcgccccaagctcagccgcctcaagacctgtgctgagctcgagatcagatcagagccagaagaacgaggtgttcctggacgtggtggaacggcttagcgtcctgatcgcctccaacgggtcgctgctgaaggtcgacgtccagggagaaatccggctgaagtcattcctcccatcgggctcagaaatgcggatcggcctgactgaggagttctgcgtgggaaagagcgaactgagaggatacggccccggcattagagtggacgaagtctccttccattcctccgtgaacctggacgagttcgagtcccaccgcatcctgcggctccaaccgccacagggggaactgaccgtgatgcgctaccagctgtccgacgatctgccgagcccccttcctttccgcttgttcccgtccgtgcaatgggataggggctccggaaggctccaagtgtaccttaagctgcgctgcgatctgctctcgaaaagccaggcgctgaacgtgcgcctgcaccttcctctgccgaggggagtggtgtccctgtcccaagagctgtcctcgcccgaacagaaggccgagctggcagagggtgctctccgctgggacctacccagagtgcagggaggctcacagctgagcggactgttccagatggacgtgcccggacctcctggacccccatcccacggtctgtccacgtccgcctcacctctgggtctggggcctgcaagcttgagcttcgaacttccgcggcatacttgctccggtctgcaagtccggttcctccggctggcgttcaggccgtgcggcaatgccaacccgcacaaatgggtccggcacctgtcgcactccgacgcttacgtgattcggatttgataagcgatcgccagaacctcgaggaccacggcgcgcctttgcggatccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagctttggacgcgtaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

(19) United States
(12) Patent Application Publication
(10) Pub. No.: US 2024/0376493 A1
(43) Pub. Date: Nov. 14, 2024

(54) OPTIMIZED AP4M1 POLYNEUCLEOTIDES AND EXPRESSION CASSETTES AND THEIR USE

(71) Applicant: THE BOARD OF REGENTS OF UNIVERSITY OF TEXAS SYSTEM, Austin, TX (US)

(72) Inventors: Steven J. GRAY, Southlake, TX (US); Xin CHEN, Irving, TX (US)

(73) Assignee: THE BOARD OF REGENTS OF UNIVERSITY OF TEXAS SYSTEM, Austin, TX (US)

(21) Appl. No.: 18/287,314

(22) PCT Filed: Apr. 21, 2022
(86) PCT No.: PCT/US2022/025749

§ 371 Date: May 15, 2024

Related U.S. Application Data
(60) Provisional application No. 63/177,559, filed on Apr. 21, 2021.

(51) Int. Cl. C12N 15/86; A61K 9/00; A61K 48/00; C07K 14/47

(52) U.S. Cl. CPC C12N 15/86; A61K 9/0019; A61K 48/005; C07K 14/47; A01K 2217/075; A01K 2227/105; C12N 2750/14143

(57) ABSTRACT

Provide herein are polynucleotides comprising a codon-optimized AP4M1 open reading frame (ORF) sequence encoding an AP4M1 polypeptide, vectors (viral or non-viral vectors) comprising the same, pharmaceutical compositions comprising the same, and methods of using the same for delivery of the polynucleotides to a cell or a subject and to treat AP4M1-related genetic disorders such as AP4M1 deficiency and spastic paraplegia type 50.

### CROSS-REFERENCE TO RELATED APPLICATIONS

This application claims benefit of priority under 35 U.S.C. § 119(e) of U.S. Provisional Application No. 63/177,559, filed Apr. 21, 2021. The disclosure of the prior application is considered part of and is herein incorporated by reference in the disclosure of this application in their entirety.

### INCORPORATION OF SEQUENCE LISTING

The material in the accompanying sequence listing is hereby incorporated by reference into this application. The accompanying sequence listing text file, name 426871-000264_SEQ_Listing_ST25, was created on Apr. 21, 2022, and is 30.01 kb. The file can be assessed using Microsoft Word on a computer that uses Windows OS.

### BACKGROUND

### Field

Provided herein are polynucleotides comprising codon-optimized AP4M1 polynucleotide sequences, vectors (viral or non-viral vectors) comprising the polynucleotides, methods of using the polynucleotides for delivery of an AP4M1 polynucleotide to a cell or a subject, and methods of using the polynucleotides for treating, e.g., spastic paraplegia type 50 (SPG50, or AP4M1-associated hereditary spastic paraplegia).

### Background Information

Hereditary spastic paraplegia (HSP) is a group of inherited diseases characterized by a progressive gait disorder, which presents with progressive spasticity and contraction in the lower limbs. HSP is also known as hereditary spastic paraparesis, familial spastic paraplegia, French settlement disease, Strumpell disease, or Strumpell-Lorrain disease. The symptoms are a result of dysfunction of long axons in the spinal cord. The affected cells are the primary motor neurons; therefore, the disease is an upper motor neuron disease. HSP can be inherited in an autosomal dominant, autosomal recessive or X-linked recessive manner. There are over 70 HSP known genotypes, and over 50 genetic loci linked to the disease.

Spastic paraplegia type 50 (SPG50) is an HSP associated disease with mutations in the AP4M1 gene, which are inherited in an autosomal recessive fashion. SPG50 is a slowly progressing neurodegenerative disorder that generally presents with global developmental delay, moderate to severe intellectual disability, impaired/absent speech, microcephaly, seizures, and progressive motor symptoms. Hypotonia develops into hypertonia, resulting in spasticity of the legs that leads to non-ambulation and wheelchair reliance. Spasticity can progress to the upper extremities, leading to the partial or total loss of use of all four limbs and torso (tetraplegia).

An AP4M1 gene encodes an AP-4 complex subunit mu-1, a subunit of the heterotetrameric AP-4 complex, which belongs to the adaptor complexes medium subunits family. This AP-4 complex is involved in the recognition and sorting of cargo proteins with tyrosine-based motifs from the trans-Golgi network to the endosomal-lysosomal system. Adaptor protein complexes are vesicle coat components involved both vesicle formation and cargo selection. The complexes control the vesicular transport of proteins in different trafficking pathways. AP-4 forms a non clathrin-associated coat on vesicles departing the trans-Golgi network and can be involved in the targeting of proteins from the trans-Golgi network to the endosomal-lysosomal system. AP-4 is also involved in protein sorting to the basolateral membrane in epithelial cells and the proper asymmetric localization of somato-dendritic proteins in neurons. Within AP-4, the mu-type subunit AP4M1 is directly involved in the recognition and binding of tyrosine-based sorting signals found in the cytoplasmic part of cargo.

There are no effective treatments for SPG50, therefore, there is a need for a new, effective therapy for treating disorders associated with AP4M1deficiency such as SPG50.

### SUMMARY

The present disclosure is based, in part, on the development of codon optimized AP4M1 genes, expression cassettes, and vectors capable of providing therapeutic levels of AP4M1 expression for treating disorders associated with AP4M1 deficiency such as spastic paraplegia 50 (e.g., SPG50, AP4M1-associated hereditary spastic paraplegia).

Thus, one aspect of the disclosure related to a polynucleotide encoding AP4M1 polypeptide, e.g., GenBank Accession XP_005250746.1, wherein the polynucleotide is codon-optimized for expression in a human cell. In one aspect, the polynucleotide encodes a human AP4M1 polypeptide.

Another aspect of the disclosure relates to an expression cassette comprising a polynucleotide encoding an AP4M1 polypeptide and vectors, transformed cells, and transgenic animals comprising the polynucleotide. In one aspect, the polynucleotide encodes a human AP4M1 polypeptide.

An aspect of the disclosure relates to recombinant adeno-associated virus (rAAV) vector comprising in 5′ to 3′ direction: a) a first AAV ITR sequence; b) a promoter sequence; c) a transgene nucleic acid molecule, wherein the transgene nucleic acid molecule comprises a nucleic acid sequence encoding an AP4M1 polypeptide; d) a polyA sequence; and e) a second AAV ITR sequence.

Another aspect of the disclosure relates to recombinant adeno-associated virus (rAAV) vector comprising in 5′ to 3′ direction: a) a first AAV ITR sequence; b) a promoter sequence; c) an intron; d) a transgene nucleic acid molecule, wherein the transgene nucleic acid molecule comprises a nucleic acid sequence encoding an AP4M1 polypeptide; e) a polyA sequence; and f) a second AAV ITR sequence.

A further aspect of the disclosure relates to a pharmaceutical composition comprising the polynucleotide, expression cassette, vector, rAAV vector and/or transformed cell described herein and a pharmaceutically acceptable carrier, such as an excipient and/or an additive.

An additional aspect relates to a method of expressing a polynucleotide encoding AP4M1 in a cell, comprising contacting the cell with the polynucleotide, expression cassette, rAAV vector and/or vector described herein, thereby expressing the polynucleotide in the cell. In one aspect, the polynucleotide encodes a human AP4M1 polypeptide. In another aspect, an AP4M1 polypeptide is a human AP4M1 polypeptide. In another aspect, an AP4M1 polynucleotide is codon-optimized for expression in human cells.

A further aspect relates to a method of expressing an AP4M1 polypeptide in a subject, comprising delivering to the subject the polynucleotide, expression cassette, vector, rAAV vector, pharmaceutical composition and/or transformed cell described herein, thereby expressing the AP4M1 polypeptide in the subject. In one aspect, the polynucleotide encodes an AP4M1 polypeptide. In another aspect, the AP4M1 polypeptide is a human AP4M1 polypeptide.

An additional aspect relates to a method of treating a disorder associated with aberrant expression of an AP4M1 gene or aberrant activity of an AP4M1 gene product in a subject in need thereof, comprising delivering to the subject a therapeutically effective amount of the polynucleotide, expression cassette, vector, rAAV vector, pharmaceutical composition and/or transformed cell described herein, thereby treating the disorder associated with aberrant expression of the AP4M1 gene or AP4M1 polypeptide in the subject.

Another aspect relates to a method of treating spastic paraplegia type 50 (SPG50) in a subject comprising delivering to the subject a therapeutically effective amount of the polynucleotide, expression cassette, vector, rAAV vector, pharmaceutical composition and/or transformed cell described herein, thereby treating SPG50 in the subject.

Therefore, the methods and compositions described herein can overcome shortcomings in the art by providing a codon-optimized AP4M1 coding sequence, expression cassettes, and vectors capable of providing therapeutic levels of AP4M1 expression for treating disorders associated with AP4M1 expression such as SPG50. These and other aspects are set forth in more detail in the description below.

### BRIEF DESCRIPTION OF THE DRAWINGS

FIG. 1 shows a map of an exemplary AP4M1 expression cassette.

FIG. 2 shows a schematic representation of a vector construct expressing human AP4M1.

FIG. 3A shows PCR results illustrating AP4M1 mRNA expression after transducing cells with AP4M1 plasmid.

FIG. 3B shows PCR results illustrating AP4M1 mRNA expression after transducing cells with AP4M1 vector.

FIG. 4 is a schematic representation of the efficacy study design.

FIG. 5A shows brain staining pictures illustrating increased hAP4M1opt mRNA expression in brain regions 3 weeks post injection.

FIG. 5B is a graph showing the RNAscope of AP4M1 mRNA in brain regions 3 weeks post injection.

FIG. 6A is a graph illustrating AAV9 capsid-induced immune response.

FIG. 6B is a graph illustrating AP4Ma peptides-induced immune response.

FIG. 7A is a graph illustrating serum toxicity in Ap4 m1 KO mice as shown by the level of aspartate transaminase (AST).

FIG. 7B is a graph illustrating serum toxicity in Ap4 m1 KO mice as shown by the level of total bilirubin (TBIL).

FIG. 7C is a graph illustrating serum toxicity in Ap4 m1 KO mice as shown by the level of albumin (ALB).

FIG. 7D is a graph illustrating serum toxicity in Ap4 m1 KO mice as shown by the level of creatine kinase (CK).

FIG. 7E is a graph illustrating serum toxicity in Ap4 m1 KO mice as shown by the level of blood urea nitrogen (BUN).

FIG. 8A is a graph illustrating body weight in Ap4 m1 KO male mice treated at p7-10.

FIG. 8B is a graph illustrating body weight in Ap4 m1 KO female mice treated at p7-10.

FIG. 8C is a graph illustrating body weight in Ap4 m1 KO male mice treated at p90.

FIG. 8D is a graph illustrating body weight in Ap4 m1 KO female mice treated at p90.

FIG. 9 is a Kaplan-Meyer graph illustrating the effects of the treatment on survival in in Ap4 m1 KO mice KO mice.

FIG. 10A is a graph illustrating hindlimb clasping score in 5 months old animals treated at p7-10.

FIG. 10B is a graph illustrating hindlimb clasping score in 5 months old animals treated at p90.

FIG. 10C is a graph illustrating total distance measured in elevated plus maze in 5 months old animals treated at p7-10.

FIG. 10D is a graph illustrating total distance measured in elevated plus maze in 5 months old animals treated at p90.

FIG. 11A is a graph illustrating hindlimb clasping score in 8 months old animals treated at p7-10.

FIG. 11B is a graph illustrating hindlimb clasping score in 8 months old animals treated at p90.

FIG. 11C is a graph illustrating total distance measured in elevated plus maze in 8 months old animals treated at p7-10.

FIG. 11D is a graph illustrating total distance measured in elevated plus maze in 8 months old animals treated at p90.

FIG. 11E is a graph illustrating total distance measured in open field in 8 months old animals treated at p7-10.

FIG. 11F is a graph illustrating total distance measured in open field in 8 months old animals treated at p90.

### DETAILED DESCRIPTION

The compositions and methods are more particularly described below and are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

Except as otherwise indicated, standard methods known to those skilled in the art can be used for production of recombinant and synthetic polypeptides, manipulation of nucleic acid sequences, production of transformed cells, the construction of rAAV constructs, modified capsid proteins, packaging vectors expressing the AAV rep and/or cap sequences, and transiently and stably transfected packaging cells. Such techniques are described in, e.g., SAMBROOK et al., MOLECULAR CLONING: A LABORATORY MANUAL 2nd Ed. (Cold Spring Harbor, NY, 1989); F. M. AUSUBEL et al. CURRENT PROTOCOLS IN MOLECULAR BIOLOGY (Green Publishing Associates, Inc. and John Wiley & Sons, Inc., New York). All publications, patent applications, patents, nucleotide sequences, amino acid sequences and other references mentioned herein are incorporated by reference in their entirety.

The present disclosure provides, inter alia, isolated polynucleotides, recombinant adeno-associated virus (rAAV) vectors, and rAAV viral vectors comprising transgene nucleic acid molecules comprising nucleic acid sequences encoding for AP4M1 polypeptides. The present disclosure also provides methods of manufacturing these isolated polynucleotides, rAAV vectors, and rAAV viral vectors, as well as their use to deliver transgenes to treat or prevent a disease or disorder, including diseases associated with loss, misfunction and/or deficiency of an AP4M1 gene.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. See, for example, Blacklowe, 1988, pp. 165-174 of Parvoviruses and Human Disease, J. R. Pattison, ed.; and Rose, Comprehensive Virology 3:1-61 (1974). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, consists essentially of, or consists of three major viral proteins: VP1, VP2 and VP3. In some aspects, AAV refers to the serotype AAV1, AAV2, AAV4, AAV5, AAV6, AAV7, AAV8, AAV9, AAV10, AAV11, AAV12, AAV13, AAVPHP.B, AAVrh74, or AAVrh.10.

Exemplary adeno-associated viruses and recombinant adeno-associated viruses include, but are not limited to all serotypes (e.g., AAV1, AAV2, AAV3, AAV4, AAV5, AAV6, AAV7, AAV8, AAV9, AAV10, AAV11, AAV12, AAV13, AAVPHP.B, AAVrh74, and AAVrh.10). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03, AAV-KP-1 (described in detail in Kerun et al. JCI Insight, 2019; 4(22):e131610) and AAV-NP59 (described in detail in Paulk et al. Molecular Therapy, 2018; 26(1): 289-303).

### AAV Structure and Function

AAV is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928. U.S. Pat. No. 9,434,928 also provides the sequences of the capsid proteins and a self-complementary genome. In one aspect, an AAV genome is a self-complementary genome. Cis-acting sequences directing viral DNA replication (rep), encapsidation/packaging, and host cell chromosome integration are contained within AAV ITRs. Three AAV promoters (named p5, p19,and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe J P, Carter B J (September 1988). “Alternate mRNA splicing is required for synthesis of adeno-associated virus VP1 capsid protein”. Journal of Virology. 62 (9): 3356-63, each of which is herein incorporated by reference. A single consensus polyA site is located at map position 95 of the AAV genome. The life cycle and genetics of AAV are reviewed in Muzyczka, Current Topics in Microbiology and Immunology, 158: 97-129 (1992).

Each VP1 protein contains a VP1 portion, a VP2 portion, and a VP3 portion. The VP1 portion is the N-terminal portion of the VP1 protein that is unique to the VP1 protein. The VP2 portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958, June 2012, the contents of which are incorporated herein by reference.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes comprise, consist essentially of, or consist of a nucleic acid molecule encoding a therapeutic protein (e.g., AP4M1) and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

### AP4M1 Polynucleotides

The present disclosure provides isolated polynucleotides comprising at least one transgene nucleic acid molecule.

In some aspects, a polynucleotide can encode a human AP4M1 polypeptide. In an aspect a polynucleotide is codon-optimized for expression in a human cell. In some aspects, a transgene nucleic acid molecule can comprise a nucleic acid sequence encoding an AP4M1 polypeptide, or at least one fragment thereof. In some aspects, a transgene nucleic acid molecule can comprise a nucleic acid sequence encoding a biological equivalent of an AP4M1 polypeptide. An AAV-AP4M1 vector is suitable for use in treating or preventing an AP4M1-associated disorder, such as spastic paraplegia type 50 (SPG50), in a subject in need thereof.

A “polynucleotide,” “nucleic acid,” or “nucleotide sequence” is a sequence of nucleotide bases; deoxyribonucleic acid (DNA) or ribonucleic acid (RNA). Nucleic acid molecules include but are not limited to genomic DNA, cDNA, mRNA, iRNA, miRNA, tRNA, ncRNA, rRNA, DNA-RNA hybrid sequences and recombinantly produced and chemically synthesized molecules such as aptamers, plasmids, antisense DNA strands, shRNA, ribozymes, nucleic acids conjugated, oligonucleotides or combinations thereof. Unless otherwise indicated, the term polynucleotide or gene includes reference to the specified sequence as well as the complementary sequence thereof. Polynucleotides can be present as a single-stranded or double-stranded and linear or covalently circularly closed molecule. As used herein, a polynucleotide can include both naturally occurring and non-naturally occurring nucleotide. A polynucleotide can be either a single or double stranded DNA sequence. A polynucleotide can comprise, for example, a gene, an open reading frame, a non-coding region, or a regulatory element. The term “open reading frame (ORF),” as used herein, refers to the portion of a polynucleotide that encodes a polypeptide.

Polynucleotides can be obtained from nucleic acid molecules present in, for example, a mammalian cell. Polynucleotides can also be synthesized in the laboratory, for example, using an automatic synthesizer. Polynucleotides can be isolated. An isolated polynucleotide can be a naturally occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid molecules naturally found immediately flanking the recombinant DNA molecule in a naturally occurring genome is removed or absent. Isolated polynucleotides also include non-naturally occurring nucleic acid molecules. “Isolated polynucleotides” can be (i) amplified in vitro, for example via polymerase chain reaction (PCR), (ii) produced recombinantly by cloning, (iii) purified, for example, by cleavage and separation by gel electrophoresis, (iv) synthesized, for example, by chemical synthesis, or (vi) extracted from a sample.

Polynucleotides can encode full-length polypeptides, polypeptide fragments, and variant or fusion polypeptides. Polynucleotides can comprise coding sequences for naturally occurring polypeptides or can encode altered sequences that do not occur in nature. Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide.

In one aspect, the polynucleotide can comprise a nucleotide sequence encoding an AP-4 complex subunit mu-1 (AP4M1) polypeptide. In one aspect, the nucleic acid sequence of the polynucleotide can comprise SEQ ID NO:5 or SEQ ID NO:40. In some aspects, a nucleic acid sequence encoding an AP4M1 polypeptide comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequence set forth in SEQ ID NOs:1 and 5 or in SEQ ID Nos:41 and 40.

In some aspects, an AP4M1 polypeptide comprises, consists essentially of, or consists of an amino acid sequence at least 65%, 70%, 75%, 80%, 85%, 90%, 95%, 96%, 97%, 98%, 99% or 100% (or any percentage in between) identical to the amino acid sequence set forth in SEQ ID NO:5, SEQ ID NO:40, or a fragment thereof. In some aspects, an AP4M1 polypeptide comprises, consists essentially of, or consists of an amino acid sequence at least 65%, 70%, 75%, 80%, 85%, 90%, 95%, 96%, 97%, 98%, 99% or 100% (or any percentage in between) identical to at least one portion of the amino acid sequence set forth in SEQ ID NO:5, SEQ ID NO:40, or a fragment thereof. In some aspects, the fragment is a functional fragment, e.g., a fragment that retains at least one function of wildtype AP4M1.

In another aspect, the nucleic acid sequence of the polynucleotide can encode an AP4M1 polypeptide. The AP4M1 polypeptide sequences can be found in GenBank, which include but are not limited to, Accession Nos: NP_004713.2, NM_004722.3.

In another aspect, the nucleotide sequence is codon-optimized for expression in a cell. By “codon-optimized,” it is meant that the coding sequence is optimized relative to a wild type coding sequence (e.g., a naturally-occurring coding sequence for AP4M1) to increase expression of the coding sequence by substituting one or more codons normally present in the coding sequence with a codon for the same (synonymous) amino acid, and/or to allow or enhance transgene detection, as codon-optimization can allow to clearly and easily distinguish a transgene from a patient's endogenous DNA and RNA sequences using molecular methods. Codon-optimization can, in some aspects, enable clear discrimination of the transgene from the endogenous AP4M1 sequence by standard molecular methods, since the sequences are different. This provides an advantage in the development of drugs and detection in preclinical or clinical specimens. For example, detection of the transgene in biopsied tissue or in blood, feces, urine, saliva, or tears. In some aspects, the substitutions minimize rare codons (e.g., human codons), increase total GC content, decrease CpG content, remove cryptic splice donor or acceptor sites, and/or add or remove ribosomal entry sites, such as Kozak sequences.

A cell includes but is not limited to a prokaryotic cell, a eukaryotic cell, an insect cell, a human cell, a rat cell, a mouse cell, a dog cell, or any mammal or plant cells. In one aspect, the cell is a human cell. In another aspect, a polynucleotide encodes an AP4M1 polypeptide from a human, rat, mouse, bovine, or any other species. In another aspect, a polynucleotide encodes a human AP4M1 polypeptide. The polynucleotide is an AP4M1 polynucleotide that encodes an AP4M1 polypeptide.

In some aspects, a codon optimized nucleic acid sequence encoding an AP4M1 polypeptide, such as that set forth in SEQ ID NO:5 or SEQ ID NO:40, can have a GC content that differs from the GC content of the wildtype human nucleic acid sequence encoding the AP4M1 polypeptide. In some aspects, the GC content of a codon optimized nucleic acid sequence encoding an AP4M1 polypeptide is more evenly distributed across the entire nucleic acid sequence, as compared to the wildtype human nucleic acid sequence encoding the AP4M1 polypeptide. Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

Furthermore, in another aspect, a CG-depleted genome can be less immunogenic when delivered in vivo. This can lead to lower immunogenicity, increased safety, and enhanced transgene expression.

In some aspects, the codon optimized nucleic acid sequence encoding an AP4M1 polypeptide, such as that set forth in SEQ ID NO:5 or SEQ ID NO:40, exhibits at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence encoding an AP4M1 polypeptide.

In some aspects, a nucleic acid sequence encoding a AP4M1 polypeptide can have the number of CpG dinucleotides reduced by 1, 2, 3, 4, 5, 10 or more CpG dinucleotides. CpG nucleotides are located within regions of DNA where a cytosine nucleotide is followed by a guanine nucleotide in the linear sequence of bases along its 5′→3′ direction. A nucleic acid sequence that encodes for a AP4M1 polypeptide but has had the number of CpG dinucleotides reduced is a CpG dinucleotide optimized AP4M1 nucleic acid sequence. A CpG dinucleotide optimized AP4M1 nucleic acid sequence can comprise, consist essentially of, or consist of a nucleic acid sequence that is no more than 65%, 70%, 75%, 80%, 85%, 90%, 95%, 96%, 97%, 98%, 99% (or any percentage in between) identical to a wildtype human nucleic acid sequence encoding a AP4M1 polypeptide. As used herein, a “wildtype human nucleic acid sequence encoding a AP4M1 polypeptide” refers to a nucleic acid sequence that encodes a healthy AP4M1 polypeptide in a human genome. An exemplary CpG dinucleotide optimized AP4M1 nucleic acid sequence is set forth in SEQ ID NO:5 and SEQ ID NO:40.

In some aspects, a CpG dinucleotide optimized AP4M1 nucleic acid sequence encoding a AP4M1 polypeptide, such as shown in SEQ ID NO:5 and SEQ ID NO:40, can comprise no donor splice sites. In some aspects, a CpG dinucleotide optimized AP4M1 nucleic acid sequence encoding a AP4M1 polypeptide can comprise no more than about one, or about two, or about three, or about four, or about five, or about six, or about seven, or about eight, or about nine, or about ten donor splice sites. In some aspects, a CpG dinucleotide optimized AP4M1 nucleic acid sequence encoding a AP4M1 polypeptide comprises at least one, or at least two, or at least three, or at least four, or at least five, or at least six, or at least seven, or at least eight, or at least nine, or at least ten fewer donor splice sites as compared to a wildtype human nucleic acid sequence encoding a AP4M1 polypeptide. Without wishing to be bound by theory, the removal of donor splice sites in the optimized nucleic acid sequence can unexpectedly and unpredictably increase expression of the AP4M1 polypeptide in vivo, as cryptic splicing is prevented. Moreover, cryptic splicing may vary between different subjects, meaning that the expression level of the AP4M1 polypeptide comprising donor splice sites may unpredictably vary between different subjects.

In some aspects, the CpG dinucleotide optimized AP4M1 nucleic acid sequence encoding a AP4M1 polypeptide, such as shown SEQ ID NO:5 and SEQ ID NO:40, has increased potency and increased safety, because it reduces overexpression of the AP4M1 gene.

In some aspects, an AP4M1 polypeptide can further comprise a protein tag. Without wishing to be bound by theory, the inclusion of a protein tag can allow for the detection and/or visualization of the exogenous AP4M1 polypeptide. As would be appreciated by the skilled artisan, non-limiting examples of protein tags include Myc tags, poly-histidine tags, FLAG-tags, HA-tags, SBP-tags or any other protein tag known in the art.

In some aspects, a codon optimized nucleic acid sequence encoding a human AP4M1 polypeptide such as SEQ ID NO:5 or SEQ ID NO:40, can comprise no donor splice sites. In some aspects, a codon optimized nucleic acid sequence encoding an AP4M1 polypeptide can comprise no more than about one, or about two, or about three, or about four, or about five, or about six, or about seven, or about eight, or about nine, or about ten donor splice sites. In some aspects, a codon optimized nucleic acid sequence encoding an AP4M1 polypeptide comprises at least one, or at least two, or at least three, or at least four, or at least five, or at least six, or at least seven, or at least eight, or at least nine, or at least ten fewer donor splice sites as compared to the wildtype human nucleic acid sequence of an AP4M1 polypeptide. Without wishing to be bound by theory, the removal of donor splice sites in the codon optimized nucleic acid molecule can unexpectedly and unpredictably increase expression of the AP4M1 polypeptide in vivo, as cryptic splicing is prevented. Moreover, cryptic splicing may vary between different subjects, meaning that the expression level of the AP4M1 polypeptide comprising donor splice sites may unpredictably vary between different subjects. Such unpredictability is unacceptable in the context of human therapy. Accordingly, a codon optimized nucleic acid sequence that that has fewer, or no donor splice sites unexpectedly and surprisingly allows for increased expression of the AP4M1 polypeptide in human subjects and regularizes expression of the AP4M1polypeptide across different human subjects.

In some aspects, a codon optimized nucleic acid sequence encoding an AP4M1 polypeptide, such as SEQ ID NO:5 and SEQ ID NO:40, can have a GC content that differs from the GC content of the wildtype human nucleic acid sequence of an AP4M1 polypeptide. In some aspects, the GC content of a codon optimized nucleic acid sequence encoding an AP4M1 polypeptide is more evenly distributed across the entire nucleic acid sequence, as compared to the wildtype human nucleic acid sequence of a AP4M1 polypeptide. Without wishing to be bound by theory, by more evenly distributing the GC content across the entire nucleic acid molecule, the codon optimized nucleic acid molecule exhibits a more uniform melting temperature (“Tm”) across the length of the transcript. The uniformity of melting temperature can result unexpectedly in increased expression of the codon optimized nucleic acid in a human subject, as transcription and/or translation of the nucleic acid occurs with less stalling of the polymerase and/or ribosome.

In some aspects, a codon optimized nucleic acid molecule encoding an AP4M1 polypeptide, such as SEQ ID NO:5 and SEQ ID NO:40, can have fewer repressive microRNA target binding sites as compared to the wildtype human nucleic acid sequence of an AP4M1 polypeptide. In some aspects, a codon optimized nucleic acid molecule encoding an AP4M1 polypeptide can have at least one, or at least two, or at least three, or at least four, or at least five, or at least six, or at least seven, or at least eight, or at least nine, or at least ten, or at least ten fewer repressive microRNA target binding sites as compared to the wildtype human nucleic acid sequence of an AP4M1 polypeptide. Without wishing to be bound by theory, by having fewer repressive microRNA target binding sites, a codon optimized nucleic acid sequence encoding an AP4M1 polypeptide can unexpectedly exhibit increased expression in a human subject.

In some aspects, a codon optimized nucleic acid sequence encoding an AP4M1 polypeptide exhibits at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence encoding an AP4M1 polypeptide.

In another aspect, a polynucleotide can have at least about 80, 85, 90, 95, 98, 99% or more identity to SEQ ID NO:5 or to SEQ ID NO:40.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some aspects the length of a reference sequence (e.g., SEQ ID NO:5 and SEQ ID NO:40) aligned for comparison purposes is at least 80% of the length of the comparison sequence, and in some aspects is at least 90% or 100%. In an aspect, the two sequences are the same length.

Ranges of desired degrees of sequence identity are approximately 80% to 100% and integer values in between. Percent identities between a disclosed sequence and a claimed sequence can be at least 80%, at least 83%, at least 85%, at least 90%, at least 95%, at least 98%, at least 99%, at least 99.5%, or at least 99.9%. In general, an exact match indicates 100% identity over the length of the reference sequence (e.g., SEQ ID NO:1 or 5, SEQ ID NO:41 or 40).

Polypeptides and polynucleotides that are sufficiently similar to polypeptides and polynucleotides described herein (e.g., AP4M1 polynucleotide or polypeptide) can be used herein. Polypeptides and polynucleotides that are about 90, 91, 92, 93, 94 95, 96, 97, 98, 99 99.5% or more identical to polypeptides and polynucleotides described herein can also be used herein.

For example, a polynucleotide can have 80% 85%, 90%, 91%, 92%, 93%, 94%, 95%, 96%, 97%, 98%, 99% or more identity to SEQ ID NO:5 or SEQ ID NO:40.

### Expression Cassette

A recombinant construct is a polynucleotide having heterologous polynucleotide elements. Recombinant constructs include expression cassettes or expression constructs, which refer to an assembly that is capable of directing the expression of a polynucleotide or gene of interest. Expression cassettes can be circular or linear nucleic acid molecules comprising one or more polynucleotides sequences in operable linkage. An aspect relates to an expression cassette comprising a polynucleotide encoding a human AP4M1 polypeptide.

An expression cassette generally includes regulatory elements operably linked to (so as to direct transcription of) a polynucleotide of interest. The regulatory elements (i.e., promoters, enhancers, transcriptional regulatory regions, translational regulatory regions, translational termination regions, and the like) and/or the polynucleotide can be native/analogous to the host cell or to each other. Alternatively, the regulatory elements can be heterologous to the host cell or to each other. See, U.S. Pat. No. 7,205,453 and U.S. Patent Application Publication Nos. 2006/0218670 and 2006/0248616. The expression cassette or recombinant construct can additionally contain one or more selectable marker genes. Methods for preparing polynucleotides operably linked to a regulatory element and expressing polypeptides in a host cell are described in, e.g., U.S. Pat. No. 4,366,246. A polynucleotide can be operably linked when it is positioned adjacent to or close to one or more regulatory elements, which direct transcription and/or translation of the polynucleotide.

In one aspect, a polynucleotide can be operably linked to a promoter.

A promoter is a nucleotide sequence that is capable of controlling the expression of a coding sequence or gene. Promoters are generally located 5′ of the sequence that they regulate. Promoters can be derived in their entirety from a native gene or be composed of different elements derived from promoters found in nature, and/or comprise synthetic nucleotide segments. Those skilled in the art will readily ascertain that different promoters can regulate expression of a coding sequence or gene in response to a particular stimulus, e.g., in a cell- or tissue-specific manner, in response to different environmental or physiological conditions, or in response to specific compounds. Promoters are typically classified into two classes: inducible and constitutive. A constitutive promoter refers to a promoter that allows for continual transcription of the coding sequence or gene under its control. An inducible promoter refers to a promoter that initiates increased levels of transcription of the coding sequence or gene under its control in response to a stimulus or an exogenous environmental condition.

A promoter can be any polynucleotide that shows transcriptional activity in a chosen host cell. A promoter can be naturally occurring, can be composed of portions of various naturally occurring promoters, or can be partially or totally synthetic. Guidance for the design of promoters can be derived from studies of promoter structure, such as that of Harley and Reynolds, Nucleic Acids Res., 15, 2343-61 (1987). In addition, the location of the promoter relative to the transcription start can be optimized. Many suitable promoters for use in mammalian cells are well known in the art, as are polynucleotides that enhance expression of an associated expressible polynucleotide. Non-limiting examples of constitutive promoters that can be used to in the present expression cassette can include cytomegalovirus (CMV) promoter and the Rous sarcoma virus promoter, chicken beta actin promoter, or ubiquitin promoter, which allow for unregulated expression in mammalian cells.

In some aspects, the promoter can be a JeT promoter or an UsP promoter. In one aspect, the promoter can comprise SEQ ID NO:3 or SEQ ID NO:39.

In another aspect, a polynucleotide can be operably linked to an intron or intervening sequence.

The presence of an optional intron or intervening sequence, placed between a promoter and a transgene (e.g., AP4M1) in an expression cassette, can increase transgene expression. A variety of introns are known in the art. Non-limiting examples of introns that can be used in the expression cassette include: MVM (minute virus of mice) intron, Factor IX truncated intron, β-globin splice donor (SD)/immunoglobin heavy chain splice acceptor (SA), Adenovirus SD/immunoglobulin SA, SV40 late SD/SA, and hybrid adenovirus SD/IgG SA.

In one aspect, the intron can comprise SEQ ID NO:4.

In another aspect, a polynucleotide can be operably linked to a polyadenylation signal. A polyadenylation signal is a short polynucleotide sequence that can be placed after a transgene (e.g., AP4M1) in an expression cassette; it is critical for nuclear export, translation, and mRNA stability. Therefore, the efficiency of transcript polyadenylation is important for transgene expression. Non-limiting examples of polyadenylation signals that can be used in the present expression cassette can include human growth hormone (hGH), SV40 late, SPA (synthetic polyA), bovine growth hormone (bGH), and SV40 late polyadenylation signals.

In some aspects, a polyadenylation signal can be a bovine growth hormone polyadenylation signal. In one aspect, a promoter can comprise SEQ ID NO:6.

In another aspect, a polynucleotide can comprise at least one adeno-associated virus (AAV) inverted terminal repeat (ITR).

The term “terminal repeat” or “TR” includes any viral terminal repeat or synthetic sequence that forms a hairpin structure and functions as an inverted terminal repeat (ITR) (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or the SV40 hairpin that serves as the origin of SV40 replication can be used as a TR, which can further be modified by truncation, substitution, deletion, insertion and/or addition. Furthermore, the TR can be partially or completely synthetic, such as the “double-D sequence” as described in U.S. Pat. No. 5,478,745 to Samulski et al.

Parvovirus genomes have palindromic sequences at both their 5′ and 3′ ends. The palindromic nature of the sequences leads to the formation of a hairpin structure that is stabilized by the formation of hydrogen bonds between the complementary base pairs. This hairpin structure is believed to adopt a “Y” or a “T” shape. See, e.g., FIELDS et al., VIROLOGY, volume 2, chapters 69 & 70 (4th ed., Lippincott-Raven Publishers).

An “AAV terminal repeat” or “AAV TR” can be from any AAV, including but not limited to serotypes 1, 2, 3, 4, 5, 6, 7, 8, 9, 10 or 11 or any other AAV now known or later discovered. In some aspects, the AAV terminal repeat does not have the native terminal repeat sequence (e.g., a native AAV TR sequence can be altered by insertion, deletion, truncation and/or missense mutations), when the terminal repeat mediates the desired functions, e.g., replication, virus packaging, integration, and/or provirus rescue, and the like. In another aspect, the AAV terminal repeat has the native terminal repeat sequence.

In one aspect, an expression cassette comprises two AAV ITRs. In some aspects, the two AAV ITRs are identical. In other aspects, the two AAV ITRs are not identical. In one aspect, the AAV ITRs can comprise SEQ ID NO:2 or 7. In one aspect, a first AAV ITR can comprise a wild-type (WT) AAV ITR, and a second AAV ITR can comprise a mutant self-complementary AAV ITR.

For example, an expression cassette can comprise two AAV ITRs wherein a first AAV ITR can comprise SEQ ID NO:2, and a second AAV ITR can comprise SEQ ID NO:2; an expression cassette can comprise two AAV ITRs wherein a first AAV ITR can comprise SEQ ID NO:7, and a second AAV ITR can comprise SEQ ID NO:7; an cassette can comprise two AAV ITRs wherein a first AAV ITR can comprise SEQ ID NO:2, and a second AAV ITR can comprise SEQ ID NO:7; or an expression cassette can comprise two AAV ITRs wherein a first AAV ITR can comprise SEQ ID NO:7, and a second AAV ITR can comprise SEQ ID NO:2.

In one aspect, an expression cassette can comprise a promoter, a human AP4M1 polynucleotide, and a polyadenylation site. For example, an expression cassette can comprise a JeT promoter, a human AP4M1 polynucleotide, and a bovine growth hormone polyadenylation signal. In one aspect, an expression cassette can comprise SEQ ID NO:3, SEQ ID NO:5, and SEQ ID NO:6. In another example, an expression cassette can comprise an UsP promoter, a human AP4M1 polynucleotide, and a bovine growth hormone polyadenylation signal. In one aspect, an expression cassette can comprise SEQ ID NO:39, SEQ ID NO:40, and SEQ ID NO:6.

In one aspect, an expression cassette can comprise a promoter, an intron, a human AP4M1 polynucleotide, and a polyadenylation site. For example, an expression cassette can comprise a JeT promoter, an intron, a human AP4M1 polynucleotide, and a bovine growth hormone polyadenylation signal. In one aspect, an expression cassette can comprise SEQ ID NO:3, SEQ ID NO:4, SEQ ID NO:5, and SEQ ID NO:6.

In another aspect, an expression cassette can comprise an AAV ITR, a promoter, a human AP4M1 polynucleotide, a polyadenylation site, and an AAV ITR. For example, an expression cassette can comprise a first AAV ITR, a JeT promoter, an intron, a human AP4M1 polynucleotide, a bovine growth hormone polyadenylation signal, and a second AAV ITR. In one aspect, an expression cassette can comprise SEQ ID NO:2, SEQ ID NO:3, SEQ ID NO:5, SEQ ID NO:6, and SEQ ID NO:7. In another example, an expression cassette can comprise a first AAV ITR, an UsP promoter, a human AP4M1 polynucleotide, a bovine growth hormone polyadenylation signal, and a second AAV ITR. In one aspect, an expression cassette can comprise SEQ ID NO:2, SEQ ID NO:39, SEQ ID NO:40, SEQ ID NO:6, and SEQ ID NO:7.

In another aspect, an expression cassette can comprise an AAV ITR, a promoter, an intron, a human AP4M1 polynucleotide, a polyadenylation site, and an AAV ITR. For example, an expression cassette can comprise a first AAV ITR, a JeT promoter, an intron, a human AP4M1 polynucleotide, a bovine growth hormone polyadenylation signal, and a second AAV ITR. In one aspect, an expression cassette can comprise SEQ ID NO:2, SEQ ID NO:3, SEQ ID NO:4, SEQ ID NO:5, SEQ ID NO:6, and SEQ ID NO:7.

In one aspect, an expression cassette can comprise SEQ ID NO:1, SEQ ID NO:42, or a sequence at least about 90% identical thereto. For example, an expression cassette can comprise a sequence 90%, 91%, 92%, 93%, 94%, 95%, 96%, 97%, 98%, 99%, or more identical to SEQ ID NO:1 or SEQ ID NO:41.

In another aspect, the expression cassette can be a self-complementary AAV genome.

A “recombinant AAV vector genome” or “rAAV genome” is an AAV genome (i.e., vDNA) that comprises at least one inverted terminal repeat (e.g., one, two or three inverted terminal repeats) and one or more heterologous nucleotide sequences. rAAV vectors generally retain the 145 base terminal repeat(s) (TR(s)) in cis to generate virus; however, modified AAV TRs and non-AAV TRs including partially or completely synthetic sequences can also serve this purpose. All other viral sequences are dispensable and can be supplied in trans (Muzyczka, (1992) Curr. Topics Microbiol. Immunol. 158:97). The rAAV vector optionally comprises two TRs (e.g., AAV TRs), which generally will be at the 5′ and 3′ ends of the heterologous nucleotide sequence(s) but need not be contiguous thereto. The TRs can be the same or different from each other. The vector genome can also contain a single ITR at its 3′ or 5′ end.

An “rAAV vector” as used herein refers to a vector comprising, consisting essentially of, or consisting of one or more transgene nucleic acid molecules and one or more AAV inverted terminal repeat sequences (ITRs). Such AAV vectors can be replicated and packaged into infectious viral particles when present in a host cell that provides the functionality of rep and cap gene products; for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing rAAV vectors may also contain elements for manufacturing purposes, e.g., antibiotic resistance genes, origin of replication sequences etc., but these are not encapsidated and thus do not form part of the AAV particle.

In some aspects, an rAAV vector can comprise at least one transgene nucleic acid molecule. In some aspects, an rAAV vector can comprise at least one AAV inverted terminal (ITR) sequence. In some aspects, an rAAV vector can comprise at least one promoter sequence. In some aspects, an rAAV vector can comprise at least one enhancer sequence. In some aspects, an rAAV vector can comprise at least one polyA sequence. In some aspects, an rAAV vector can comprise a RepCap sequence.

In some aspects, an rAAV vector can comprise a first AAV ITR sequence, a promoter sequence, a transgene nucleic acid molecule and a second AAV ITR sequence. In some aspects, an rAAV vector can comprise, in the 5′ to 3′ direction, a first AAV ITR sequence, a promoter sequence, an intron sequence, a transgene nucleic acid molecule and a second AAV ITR sequence.

In some aspects, an rAAV vector can comprise a first AAV ITR sequence, a promoter sequence, an intron sequence, a transgene nucleic acid molecule, a polyA sequence and a second AAV ITR sequence. In some aspects, an rAAV vector can comprise, in the 5′ to 3′ direction, a first AAV ITR sequence, a promoter sequence, an intron sequence, a transgene nucleic acid molecule, a polyA sequence and a second AAV ITR sequence.

In some aspects, an rAAV vector can comprise more than one transgene nucleic acid molecule. In some aspects, an rAAV vector can comprise at least two transgene nucleic acid molecules, such that the rAAV vector comprises a first transgene nucleic acid molecule and an at least second transgene nucleic acid molecule. In some aspects, the first and the at least second transgene nucleic acid molecule can comprise the same nucleic acid sequence. In some aspects, the first and the at least second transgene nucleic acid molecules can comprise different nucleic acid sequences. In some aspects, the first and the at least second transgene nucleic acid sequences can be adjacent to each other.

In some aspects, an rAAV vector can comprise more than one promoter sequence. In some aspects, an rAAV vector can comprise at least two promoter sequences, such that the rAAV vector comprises a first promoter sequence and an at least second promoter sequence. In some aspects, the first and the at least second promoter sequences can comprise the same sequence. In some aspects, the first and the at least second promoter sequences can comprise different sequences. In some aspects, the first and the at least second promoter sequences can be adjacent to each other. In some aspects wherein an rAAV vector also comprises a first transgene nucleic acid molecule and an at least second transgene nucleic acid molecule, the first promoter can be located upstream (5′) of the first transgene nucleic acid molecule and the at least second promoter can be located between the first transgene nucleic acid molecule and the at least second transgene nucleic acid molecule, such that the at least second promoter is downstream (3′) of the first transgene nucleic acid molecule and upstream (5′) of the at least second transgene nucleic acid molecule.

Any of the preceding rAAV vectors can further comprise at least one enhancer. The at least one enhancer can be located anywhere in the rAAV vector. In some aspects, the at least one enhancer can be located immediately upstream (5′) of a promoter. Thus, an rAAV vector can comprise, in the 5′ to 3′ direction, a first AAV ITR sequence, an enhancer, a promoter sequence, a transgene nucleic acid molecule, a polyA sequence, and a second AAV ITR sequence. In some aspects, the at least one enhancer can be located immediately downstream (3′) of a promoter. Thus, an rAAV vector can comprise, in the 5′ to 3′ direction, a first AAV ITR sequence, a promoter sequence, an enhancer, a transgene nucleic acid molecule, a polyA sequence, and a second AAV ITR sequence. In some aspects, the at least one enhancer can be located immediately downstream of a transgene nucleic acid molecule. Thus, an rAAV vector can comprise, in the 5′ to 3′ direction, a first AAV ITR sequence, a promoter sequence, an intron sequence, a transgene nucleic acid molecule, an enhancer, a polyA sequence, and a second AAV ITR sequence.

### AAV ITR Sequences

In some aspects, an AAV ITR sequence can comprise any AAV ITR sequence known in the art. In some aspects, an AAV ITR sequence can be an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence or an AAVrh.10 ITR sequence.

Thus, in some aspects, an AAV ITR sequence can comprise, consist essentially of, or consist of an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence, or an AAVrh.10 ITR sequence. In some aspects, an AAV ITR sequence is a wildtype AAV ITR sequence. In some aspects, an AAV ITR sequence is modified (e.g., mutated) AAV ITR sequence. In some aspects, an rAAV vector described herein comprises one mutated AAV ITR and one wildtype AAV ITR.

In some aspects, an AAV ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in any one of SEQ ID NOs:2, 7 or 12-20.

In some aspects, an rAAV provided herein comprises a first and a second AAV ITR sequence, wherein the first AAV ITR sequence comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:2 and the second AAV ITR sequence comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:7.

### Promoter Sequence and Enhancers

The term “promoter” and “promoter sequence” as used herein means a control sequence that is a region of a polynucleotide sequence at which the initiation and rate of transcription of a coding sequence, such as a gene or a transgene, are controlled. Promoters can be constitutive, inducible, repressible, or tissue-specific, for example. Promoters can contain genetic elements at which regulatory proteins and molecules such as RNA polymerase and transcription factors may bind. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, a synapsin promoter, an H1 promoter, a hybrid chicken beta-actin (CBA) promoter, a ubiquitous chicken β-actin hybrid (CBh) promoter, a small nuclear RNA (U1a or U1b) promoter, an MECP2 promoter, an MeP418 promoter, an MeP426 promoter, a human variant of the MeP426 promoter, a minimal MECP2 promoter, a VMD2 promoter, an mRho promoter, or an EF1 promoter.

Additional non-limiting exemplary promoters provided herein include, but are not limited to UsP, EFla, Ubc, human β-actin, CAG, TRE, Ac5, Polyhedrin, CaMKIIa, Gall, TEF1, GDS, ADH1, Ubi, and α-1-antitrypsin (hAAT). Nucleotide sequences of promoters can be modified to increase or decrease the efficiency of mRNA transcription. For example, the TATA box of 7SK, U6 and H1 promoters can be modified to abolish RNA polymerase III transcription and stimulate RNA polymerase II-dependent mRNA transcription.

Synthetically-derived promoters can be used for ubiquitous or tissue specific expression. Furthermore, virus-derived promoters, some of which are noted above, can be useful in the methods disclosed herein, e.g., CMV, HIV, adenovirus, and AAV promoters. In some aspects, a promoter is used together with at least one enhancer to increase the transcription efficiency. Non-limiting examples of enhancers include an interstitial retinoid-binding protein (IRBP) enhancer, an RSV enhancer or a CMV enhancer.

In some aspects, a promoter sequence can comprise, consist essentially of, or consist of a Rous sarcoma virus (RSV) LTR promoter sequence (optionally with the RSV enhancer), a cytomegalovirus (CMV) promoter sequence, an SV40 promoter sequence, a dihydrofolate reductase promoter sequence, a JeT promoter sequence (SEQ ID NO:3), a MeP229 promoter (SEQ ID NO: 21), a β-actin promoter sequence, a phosphoglycerol kinase (PGK) promoter sequence, a U6 promoter sequence (SEQ ID NO: 22), synapsin I promoter (SEQ ID NO: 23), synapsin II promoter (SEQ ID NO: 24), an H1 promoter sequence, a hybrid chicken beta-actin (CBA) promoter sequence (SEQ ID NO: 25), a ubiquitous chicken β-actin hybrid (CBh) promoter sequence (SEQ ID NO: 26), a shortened synapsin promoter (hSyn) (SEQ ID NO: 27), a small nuclear RNA (U1a or U1b) promoter sequence, an MECP2 promoter sequence (SEQ ID NO: 28), an MeP418 promoter, an MeP426 promoter sequence, a small ubiquitous promoter sequence (also known as a Jet+I promoter sequence, SEQ ID NO: 29), a VMD2 promoter sequence, an mRho promoter sequence, an EFI promoter sequence, an EFla promoter sequence, a Ubc promoter sequence, a human β-actin promoter sequence, a CAG promoter sequence, a TRE promoter sequence, an Ac5 promoter sequence, a Polyhedrin promoter sequence, a CaMKIIa promoter sequence, a Gall promoter sequence, a TEF1 promoter sequence, a GDS promoter sequence, an ADH1 promoter sequence, a Ubi promoter sequence, a MeP426 promoter, an UsP promoter (SEQ ID NO:39) or an α-1-antitrypsin (hAAT) promoter sequence.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) or synthetic techniques such that transcription of that gene is directed by the linked enhancer/promoter. Non-limiting examples of linked enhancer/promoter for use in the methods, compositions and constructs provided herein include a PDE promoter plus IRBP enhancer or a CMV enhancer plus U1a promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

As used throughout the disclosure, the term “operably linked” refers to the expression of a gene (i.e., a transgene) that is under the control of a promoter with which it is spatially connected. A promoter can be positioned 5′ (upstream) or 3′ (downstream) of a gene under its control. A promoter can be positioned 5′(upstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

In some aspects, a promoter sequence can comprise, consist essentially of, or consist of a JeT promoter sequence (e.g., SEQ ID NO:3). A JeT promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:3.

In some aspects, a promoter sequence can comprise, consist essentially of, or consist of an UsP promoter sequence. An UsP promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:39.

In some aspects, a promoter sequence can comprise, consist essentially of, or consist of a JeT+I promoter sequence. A Jet+I promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:29.

In some aspects, a promoter sequence can comprise, consist essentially of, or consist of a hybrid chicken β-actin promoter sequence. A hybrid chicken β-actin promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:26.

A hybrid chicken β-actin promoter sequence can comprise a CMV sequence (e.g., SEQ ID NO:30), a chicken β-actin promoter sequence, a chicken β-actin exon 1 sequence (e.g., SEQ ID NO:31), a chicken β-actin intron 1 sequence (e.g., SEQ ID NO:32), a minute virus of mice (MVM) intron sequence (e.g., SEQ ID NO:33), or any combination thereof. In some aspects, a hybrid chicken β-actin promoter sequence can comprise, in the 5′ to 3′ direction, a CMV sequence (e.g., SEQ ID NO:30), a chicken β-actin promoter sequence, chicken β-actin exon 1 sequence, a chicken β-actin intron 1 sequence and a minute virus of mice (MVM) intron sequence.

In some aspects, a CMV sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:30. The β-actin exon 1 sequence may comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:31. The chicken β-actin intron 1 sequence may comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:32. The MVM intron sequence may comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:33.

In some aspects, a promoter sequence can comprise, consist essentially of, or consist of a U6 promoter sequence. A U6 promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:22.

In some aspects, a promoter sequence can comprise a shortened version of a synapsin promoter sequence (hSyn promoter) (SEQ ID NO:27) or a rat MeCP2 promoter SEQ ID NO:28. A synapsin promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:23. A synapsin promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence put forth in SEQ ID NO:24.

### Transgene Nucleic Acid Molecules

Transgene nucleic acid molecules can comprise, consist essentially of, or consist of any of the transgene nucleic acid molecules described above under the heading “isolated polynucleotides comprising transgene sequences”.

In some aspects, a transgene nucleic acid molecule present in an rAAV vector can be under transcriptional control of a promoter sequence also present in the same rAAV vector.

polyA Sequences

In some aspects, a polyadenylation (polyA) sequence can comprise any polyA sequence known in the art. The polyA sequence may be a synthetic polyA sequence or a polyA sequence derived from a naturally occurring protein. Non-limiting examples of polyA sequences include, but are not limited to, an MECP2 polyA sequence, a retinol dehydrogenase 1 (RDH1) polyA sequence, a bovine growth hormone (BGH) polyA sequence (SEQ ID NO:6), an SV40 polyA sequence (e.g., SEQ ID NO:34), a SPA49 polyA sequence, a sNRP-TK65 polyA sequence, a sNRP polyA sequence, or a TK65 polyA sequence.

Thus, a polyA sequence can comprise, consist essentially of, or consist of an MeCP2 polyA sequence, a retinol dehydrogenase 1 (RDH1) polyA sequence, a bovine growth hormone (BGH) polyA sequence, an SV40 polyA sequence, a SPA49 polyA sequence, a sNRP-TK65 polyA sequence, a sNRP polyA sequence, or a TK65 polyA sequence.

In some aspects, a polyA sequence can comprise, consist essentially of, or consist of a bovine growth hormone (BGH) polyA sequence. In some aspects, a bovine growth hormone (BGH) polyA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical the sequence set forth in SEQ ID NO:6.

In some aspects, a polyA sequence can comprise, consist essentially of, or consist of an SV40 pA sequence. In some aspects, an SV40 pA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical the sequence set forth in SEQ ID NO:34.

In certain aspects, an rAAV vector disclosed herein comprises a Kozak sequence. In some aspects, an Kozak sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence set forth in SEQ ID NO:35.

In certain aspects, an rAAV vector disclosed herein comprises a Woodchuck Hepatitis Virus (WHV) Posttranscriptional Regulatory Element (WPRE). In some aspects, a WPRE sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence set forth in SEQ ID NO:36.

In certain aspects, an rAAV vector described herein comprises, in 5′ to 3′ order, a first AAV2 ITR of SEQ ID NO:2; a Jet promoter of SEQ ID NO:3; a codon-optimized AP4M1 sequence of SEQ ID NO:5; a BGH polyA sequence of SEQ ID NO:6; and a second AAV2 ITR of SEQ ID NO:7.

In other aspects, an rAAV vector described herein comprises, in 5′ to 3′ order, a first AAV2 ITR of SEQ ID NO:2; an UsP promoter of SEQ ID NO:39; a codon-optimized AP4M1 sequence of SEQ ID NO:40; a BGH polyA sequence of SEQ ID NO:6; and a second AAV2 ITR of SEQ ID NO:7.

### Bacterial Plasmids

In some aspects, the rAAV vectors of the present disclosure can be contained within a bacterial plasmid to allow for propagation of the rAAV vector in vitro. Thus, the present disclosure provides bacterial plasmids comprising any of the rAAV vectors described herein. A bacterial plasmid can further comprise an origin of replication sequence. A bacterial plasmid can further comprise an antibiotic resistance gene. A bacterial plasmid can further comprise a resistance gene promoter. A bacterial plasmid can further comprise a prokaryotic promoter. In some aspects, a bacterial plasmid of the present disclosure can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the nucleic acid sequence put forth in SEQ ID NO:1 or SEQ ID NO:41.

### Origin of Replication Sequence

In some aspects, an origin of replication sequence can comprise, consist essentially of, or consist of any origin of replication sequence known in the art. The origin of replication sequence can be a bacterial origin of replication sequence, thereby allowing the rAAV vector comprising said bacterial origin of replication sequence to be produced, propagated and maintained in bacteria, using methods standard in the art.

### Antibiotic Resistance Genes

In some aspects, bacterial plasmids, rAAV vectors and/or rAAV viral vectors of the disclosure can comprise an antibiotic resistance gene.

In some aspects, an antibiotic resistance gene can comprise, consist essentially of, or consist of any antibiotic resistance genes known in the art. Examples of antibiotic resistance genes known in the art include, but are not limited to kanamycin resistance genes, spectinomycin resistance genes, streptomycin resistance genes, ampicillin resistance genes, carbenicillin resistance genes, bleomycin resistance genes, erythromycin resistance genes, polymyxin B resistance genes, tetracycline resistance genes and chloramphenicol resistance genes.

In some aspects, an antibiotic resistance gene can be a kanamycin resistance gene. In some aspects, a kanamycin resistance gene can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the nucleic acid sequence put forth in SEQ ID NO:37.

### Resistance Gene Promoter

In some aspects, bacterial plasmids, rAAV vectors and/or rAAV viral vectors of the disclosure can comprise a resistance gene promoter. In some aspects, a resistance gene promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the nucleic acid sequence put forth in SEQ ID NO:38.

### Vector

An expression cassette can be delivered to cells (e.g., a plurality of different cells or cell types including target cells or cell types and/or non-target cell types) in a vector (e.g., an expression vector). A vector can be an integrating or non-integrating vector, referring to the ability of the vector to integrate the expression cassette and/or transgene into a genome of a cell. Either an integrating vector or a non-integrating vector can be used to deliver an expression cassette containing one or more polynucleotides described herein. Examples of vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs); episomal vectors; transposons (e.g., PiggyBac); and (b) viral vectors such as retroviral vectors, lentiviral vectors, adenoviral vectors, and AAV vectors. Viruses have several advantages for delivery of nucleic acids, including high infectivity and/or tropism for certain target cells or tissues. In some cases, a virus is used to deliver a nucleic acid molecule or expression cassette comprising one or more regulatory elements, as described herein, operably linked to a gene.

In some aspects, the isolated polynucleotides comprising at least one transgene nucleic acid molecule described herein can be a recombinant AAV (rAAV) vector.

As used herein, the term “vector” refers to a nucleic acid comprising, consisting essentially of, or consisting of an intact replicon such that the vector may be replicated when placed within a cell, for example by a process of transfection, infection, or transformation. A vector can comprise a signal peptide or signal polypeptide. It is understood in the art that once inside a cell, a vector may replicate as an extrachromosomal (episomal) element or may be integrated into a host cell chromosome. Vectors may include nucleic acids derived from retroviruses, adenoviruses, herpesvirus, baculoviruses, modified baculoviruses, papovaviruses, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, consisting essentially of, or consisting of DNA condensed with cationic polymers such as heterogeneous polylysine, defined-length oligopeptides, and polyethyleneimine, in some cases contained in liposomes; and the use of ternary complexes comprising, consisting essentially of, or consisting of a virus and polylysine-DNA.

With respect to general recombinant techniques, vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of cloned transgenes to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

An aspect relates to a vector comprising one or more polynucleotides described herein. In one aspect, the vector can be a viral vector. In some aspects, the vector can be an AAV vector.

Parvovirus encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, HI parvovirus, muscovy duck parvovirus, snake parvovirus, and B19 virus. Other autonomous parvoviruses are known to those skilled in the art. See, e.g., FIELDS et al., VIROLOGY, volume 2, chapter 69 (4^{th} ed., Lippincott-Raven Publishers).

The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIELDS et al., VIROLOGY, volume 2, chapter 69 (4^{th} ed., Lippincott-Raven Publishers).

The term “adeno-associated virus” (AAV) includes without limitation AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. See, e.g., BERNARD N. FIELDS et al., VIROLOGY, volume 2, chapter 69 (4th ed., Lippincott-Raven Publishers). A number of additional AAV serotypes and clades have been identified (see, e.g., Gao et al., (2004) 1 Virol. 78:6381-6388), which are also encompassed by the term “AAV.”

A “viral vector” is defined as a recombinantly produced virus or viral particle that contains a polynucleotide to be delivered into a host cell, either in vivo, ex vivo or in vitro. Examples of viral vectors include retroviral vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, e.g., Schlesinger and Dubensky (1999) Curr. Opin. Biotechnol. 5:434-439 and Ying, et al. (1999) Nat. Med. 5(7):823-827.

An “AAV virion” or “AAV viral particle” or “AAV viral vector” or “rAAV viral vector” or “AAV vector particle” or “AAV particle” refers to a viral particle composed of at least one AAV capsid protein and an encapsidated polynucleotide rAAV vector. Thus, production of an rAAV viral vector necessarily includes production of an rAAV vector, as such a vector is contained within an rAAV vector.

As used herein, the term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1:VP2:VP3) or 1:1:20 (VP1:VP2:VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz J E, Samulski R J (December 2000). “Building a better vector: the manipulation of AAV virions”. Virology. 278 (2): 301-8, each of which is incorporated herein by reference in its entirety.

The present disclosure provides an rAAV viral vector comprising: a) any of the rAAV vectors described herein, or complement thereof; and b) an AAV capsid protein.

The present disclosure provides an rAAV viral vector comprising: a) any of the rAAV vectors described herein; and b) an AAV capsid protein.

An AAV capsid protein can be any AAV capsid protein known in the art. An AAV capsid protein can be an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein.

In one aspect, a vector can be an AAV vector and the AAV vector can comprise a promoter, an intron, a human AP4M1 polynucleotide, and a polyadenylation site. For example, a vector can comprise SEQ ID NO:3, SEQ ID NO:4, SEQ ID NO:5, and SEQ ID NO:6.

In another aspect, a vector can be an AAV vector and the AAV vector can comprise a promoter, a human AP4M1 polynucleotide, and a polyadenylation site. For example, a vector can comprise SEQ ID NO:39, SEQ ID NO:40, and SEQ ID NO:6.

In one aspect, a vector can be an AAV vector and the AAV vector can comprise a first AAV ITR, a promoter, an intron, a human AP4M1 polynucleotide, a polyadenylation site, and a second AAV ITR. For example, a vector can comprise SEQ ID NO:2, SEQ ID NO:3, SEQ ID NO:4, SEQ ID NO:5, SEQ ID NO:6, and SEQ ID NO:7.

In one aspect, a vector can be an AAV vector and the AAV vector can comprise a first AAV ITR, a promoter, a human AP4M1 polynucleotide, a polyadenylation site, and a second AAV ITR. For example, a vector can comprise SEQ ID NO:2, SEQ ID NO:39, SEQ ID NO:40, SEQ ID NO:6, and SEQ ID NO:7.

In one aspect, a vector can be an AAV vector and the AAV vector can comprise SEQ ID NO:1 or a sequence at least about 90% identical thereto. For example, a vector can comprise a sequence 90%, 91%, 92%, 93%, 94%, 95%, 96%, 97%, 98%, 99%, or more identical to SEQ ID NO: 1.

In another aspect, a vector can be an AAV vector and the AAV vector can comprise SEQ ID NO:41 or a sequence at least about 90% identical thereto. For example, a vector can comprise a sequence 90%, 91%, 92%, 93%, 94%, 95%, 96%, 97%, 98%, 99%, or more identical to SEQ ID NO:41.

The virus vectors described herein can further be “targeted” virus vectors (e.g., having a directed tropism) and/or a “hybrid” parvovirus (i.e., in which the viral ITRs and viral capsid are from different parvoviruses) as described in, for example, international patent publication WO 00/28004 and Chao et al., (2000) Mol. Therapy 2:619.

Furthermore, the viral capsid or genomic elements can contain other modifications, including insertions, deletions and/or substitutions.

In one aspect, the AAV vector comprises wild-type capsid proteins. In another aspect, the AAV vector comprises a modified capsid protein.

Alternative rAAV Vector and rAAV Viral Vector Embodiments
1. A recombinant adeno-associated virus (rAAV) vector comprising in 5′ to 3′ direction:
   a) a first AAV ITR sequence;
   b) a promoter sequence;
   c) an intron sequence
   d) a transgene nucleic acid molecule, wherein the transgene nucleic acid molecule comprises a nucleic acid sequence encoding an AP4M1 polypeptide;
   e) a polyA sequence; and
   f) a second AAV ITR sequence.
2. The rAAV vector of embodiment 1, wherein the transgene nucleic acid molecule comprises the nucleic acid sequence set forth in SEQ ID NO:5.
3. The rAAV vector of embodiment 1, wherein the vector comprises SEQ ID NO:1. In an embodiment, SEQ ID NO:2 is nucleotides 1-106 of SEQ ID NO: 1. In an embodiment, SEQ ID NO:3 is nucleotides 126-289 of SEQ ID NO:1. In an embodiment, SEQ ID NO:4 is nucleotides 298-453 of SEQ ID NO: 1. In an embodiment, SEQ ID NO:5 is nucleotides 467-1832 of SEQ ID NO:1. In an embodiment, SEQ ID NO:6 is nucleotides 1882-2106 of SEQ ID NO:1. In an embodiment, SEQ ID NO:7 is nucleotides 2141-2309 of SEQ ID NO: 1.
4. The rAAV vector of any one of the preceding embodiments, wherein the transgene nucleic acid sequence encoding for an AP4M1 polypeptide exhibits at least 5%, at least 10%, at least 20%, at least 30%, at least 50%, at least 75%, at least 100%, at least 200%, at least 300%, at least 500%, or at least 1000% increased expression in a human subject relative to a mutated AP4M1 nucleic acid sequence.
5. The rAAV vector of any one of the preceding embodiments, wherein the first AAV ITR sequence comprises the nucleic acid sequence set forth in SEQ ID NO:2.
6. The rAAV vector of any one of the preceding embodiments, wherein the second AAV ITR sequence comprises the nucleic acid sequence set forth in SEQ ID NO:7.
7. The rAAV vector of any one of the preceding embodiments, wherein the promoter sequence comprises a Rous sarcoma virus (RSV) LTR promoter (optionally with an RSV enhancer), a cytomegalovirus (CMV) promoter, an SV40 promoter, a dihydrofolate reductase promoter, a beta-actin promoter, a phosphoglycerol kinase (PGK) promoter, a U6 promoter, a JetI promoter, an H1 promoter, a CAG promoter, a hybrid chicken beta-actin (CBA) promoter, an MeCP2 promoter, an EF1 promoter, a ubiquitous chicken β-actin hybrid (CBh) promoter, a U1a promoter, a U1b promoter, an MeCP2 promoter, an MeP418 promoter, an MeP426 promoter, a minimal MeCP2 promoter, a VMD2 promoter, an mRho promoter, EFla promoter, an Ubc promoter, a human β-actin promoter, a synapsin (hSyn) promoter sequence, a TRE promoter, an Ac5 promoter, a Polyhedrin promoter, a CaMKIIa promoter, a Gall promoter, a TEF1 promoter, a GDS promoter, an ADH1 promoter, an Ubi promoter, a JeT promoter, an UsP promoter or an α-1-antitrypsin (hAAT) promoter.
8. The rAAV vector of any one of the preceding embodiments, wherein the promoter sequence comprises the nucleic acid sequence set forth in SEQ ID NO:3.
9. The rAAV vector of any one of the preceding embodiments, wherein the polyA sequence comprises the nucleic acid sequence set forth in SEQ ID NO:6.
10. An rAAV vector of any one of the preceding embodiments, comprising, in the 5′ to 3′ direction:
   a) a first AAV ITR sequence comprising the nucleic acid sequence set forth in SEQ ID NO:2;
   b) a promoter sequence comprising the nucleic acid sequence set forth in SEQ ID NO:3;
   c) an intron sequence comprising the nucleic acid sequence set forth in SEQ ID NO:4
   d) a transgene nucleic acid molecule, wherein the transgene nucleic acid molecule comprises a nucleic acid sequence encoding for an AP4M1 polypeptide, wherein the nucleic acid sequence encoding for an AP4M1 polypeptide comprises the nucleic acid sequence set forth in SEQ ID NO:5;
   e) a polyA sequence comprising the nucleic acid sequence set forth in SEQ ID NO:6; and
   f) a second AAV ITR sequence comprising the nucleic acid sequence set forth in SEQ ID NO:7.
11. A recombinant adeno-associated virus (rAAV) vector comprising in 5′ to 3′ direction:
   a) a first AAV ITR sequence;
   b) a promoter sequence;
   c) a transgene nucleic acid molecule, wherein the transgene nucleic acid molecule comprises a nucleic acid sequence encoding an AP4M1 polypeptide;
   d) a polyA sequence; and
   e) a second AAV ITR sequence.
12. The rAAV vector of embodiment 11, wherein the transgene nucleic acid molecule comprises the nucleic acid sequence set forth in SEQ ID NO:40.
13. The rAAV vector of embodiment 12, wherein the vector comprises SEQ ID NO:41.
14. The rAAV vector of any one of embodiments 11-13, wherein the transgene nucleic acid sequence encoding for an AP4M1 polypeptide exhibits at least 5%, at least 10%, at least 20%, at least 30%, at least 50%, at least 75%, at least 100%, at least 200%, at least 300%, at least 500%, or at least 1000% increased expression in a human subject relative to a mutated AP4M1 nucleic acid sequence.
15. The rAAV vector of any one of embodiments 11-14, wherein the first AAV ITR sequence comprises the nucleic acid sequence set forth in SEQ ID NO:2.
16. The rAAV vector of any one of embodiments 11-15, wherein the second AAV ITR sequence comprises the nucleic acid sequence set forth in SEQ ID NO:7.
17. The rAAV vector of any one of embodiments 11-16, wherein the promoter sequence comprises a Rous sarcoma virus (RSV) LTR promoter (optionally with an RSV enhancer), a cytomegalovirus (CMV) promoter, an SV40 promoter, a dihydrofolate reductase promoter, a beta-actin promoter, a phosphoglycerol kinase (PGK) promoter, a U6 promoter, a JetI promoter, an H1 promoter, a CAG promoter, a hybrid chicken beta-actin (CBA) promoter, an MeCP2 promoter, an EF1 promoter, a ubiquitous chicken β-actin hybrid (CBh) promoter, a U1a promoter, a U1b promoter, an MeCP2 promoter, an MeP418 promoter, an MeP426 promoter, a minimal MeCP2 promoter, a VMD2 promoter, an mRho promoter, EFla promoter, an Ubc promoter, a human 3-actin promoter, a synapsin (hSyn) promoter sequence, a TRE promoter, an Ac5 promoter, a Polyhedrin promoter, a CaMKIIa promoter, a Gall promoter, a TEF1 promoter, a GDS promoter, an ADH1 promoter, an Ubi promoter, a JeT promoter, an UsP promoter or an α-1-antitrypsin (hAAT) promoter.
18. The rAAV vector of any one of embodiments 11-17, wherein the promoter sequence comprises the nucleic acid sequence set forth in SEQ ID NO:39.
19. The rAAV vector of any one of embodiments 11-18, wherein the polyA sequence comprises the nucleic acid sequence set forth in SEQ ID NO:6.
20. An rAAV vector of any one of embodiments 11-19, comprising, in the 5′ to 3′ direction:
   a) a first AAV ITR sequence comprising the nucleic acid sequence set forth in SEQ ID NO:2;
   b) a promoter sequence comprising the nucleic acid sequence set forth in SEQ ID NO:39;
   c) a transgene nucleic acid molecule, wherein the transgene nucleic acid molecule comprises a nucleic acid sequence encoding for an AP4M1 polypeptide, wherein the nucleic acid sequence encoding for an AP4M1 polypeptide comprises the nucleic acid sequence set forth in SEQ ID NO:40;
   d) a polyA sequence comprising the nucleic acid sequence set forth in SEQ ID NO:6; and
   e) a second AAV ITR sequence comprising the nucleic acid sequence set forth in SEQ ID NO:7.
21. An rAAV viral vector comprising:
   (i) an AAV capsid protein; and
   (ii) an rAAV vector of any one of the preceding embodiments.
22. The rAAV viral vector of embodiment 21, wherein the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein.
23. The rAAV viral vector of embodiment 22, wherein the AAV capsid protein is an AAV9 capsid protein.
24. A pharmaceutical composition comprising:
   a) the rAAV viral vector of any one of embodiments 21-23; and at least one pharmaceutically acceptable excipient and/or additive.
25. A method for treating a subject having a disease and/or disorder involving an AP4M1 gene, the method comprising administering to the subject at least one therapeutically effective amount of the rAAV viral vector of any one of embodiments 21-23 or the pharmaceutical composition of embodiment 24.
26. The method of embodiment 25, wherein the disease and/or disorder involving an AP4M1 gene is spastic paraplegia type 50 (SPG50).
27. The method of claim 25 or embodiment 26, wherein the rAAV viral vector or the pharmaceutical composition is administered to the subject at a dose ranging from about 10¹¹ to about 10¹⁸ viral vector particles.
28. The method of embodiment 27, wherein the rAAV viral vector or the pharmaceutical composition is administered to the subject at a dose ranging from about 10¹³ to about 10¹⁶ viral vector particles.
29. The method of any one of embodiments 25-28, wherein the rAAV viral vector or the pharmaceutical composition is administered to the subject intravenously, intrathecally, intracisternal-magna, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally or intranerve.
30. The method of embodiment 29, wherein the rAAV viral vector or pharmaceutical composition is administered intrathecally.
31. The method of embodiment 29, wherein the rAAV viral vector or pharmaceutical composition is for lumbar intrathecal administration.
32. The rAAV viral vector of any one of embodiments 21-23 or the pharmaceutical composition of embodiment 24 for use in treating a disease and/or disorder involving an AP4M1 gene in a subject in need thereof.
33. The use of embodiment 32, wherein the disease and/or disorder involving an AP4M1 gene is spastic paraplegia type 50 (SPG50).
34. The use of embodiment 32 or embodiment 33, wherein the rAAV viral vector or the pharmaceutical composition is for administration to the subject at a dose ranging from about 10¹¹ to about 10¹⁸ viral vector particles.
35. The use of any of embodiments 32-34, wherein the rAAV viral vector or the pharmaceutical composition is for administration to the subject at a dose ranging from about 10¹³ to about 10¹⁶ viral vector particles.
36. The use of any one of embodiments 32-35, wherein the rAAV viral vector or the pharmaceutical composition is for administration to the subject intravenously, intrathecally, intracisternal-magna, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally or intranerve.
37. The use of embodiment 36, wherein the rAAV viral vector or pharmaceutical composition is for administration intrathecally.
38. The use of embodiment 36, wherein the rAAV viral vector or pharmaceutical composition is for lumbar intrathecal administration.

### Transformed Cell and Transgenic Animal

An aspect relates to a transformed cell comprising a polynucleotide, an expression cassette, and/or a vector described herein.

A nucleic acid construct (i.e., a polynucleotide, an expression cassette or a vector) can be introduced into a cell to be altered thus allowing expression of the recombinant protein within the cell. A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as Lipofectamine™, Dojindo Hilymax™, Fugene™, jetPEI™, Effectene™ and DreamFect™.

A viral vector can deliver a nucleic acid construct to a cell through viral transduction of the cell. As used herein, “transduction” of a cell by a virus vector (e.g., an AAV vector) means entry of the vector into the cell and transfer of genetic material into the cell by the incorporation of the polynucleotide into the virus vector and subsequent transfer into the cell via the virus vector.

In one aspect, a polynucleotide, expression cassette, and/or vector can be stably incorporated into the cell genome.

The nucleic acid construct can be introduced into a host cell to be altered thus allowing expression of the chimeric protein within the cell. A variety of host cells are known in the art and suitable for chimeric proteins expression. Examples of typical cell used for transfection include, but are not limited to, a prokaryotic cell, a eukaryotic cell, a bacterial cell, a yeast cell, an insect cell, a mammalian cell, a human cell, or a plant cell. For example, E. coli, Bacillus, Streptomyces, Pichia pastoris, Salmonella typhimurium, Drosophila S2, Spodoptera SJ9, CHO, COS (e.g., COS-7),3T3-F442A, HeLa, HUVEC, HUAEC, NIH 3T3, Jurkat, 293, 293H, or 293F.

Other examples of cells that can be modified by the introduction of the polynucleotide, expression cassette or vector described herein can include any cell into which an AP4M1 gene is sought to be expressed, regardless of the purpose of the modification (e.g., research purpose, pre-clinical or clinical purpose, etc.). The transformed cell may be an in vitro, ex vivo or in vivo cell. In some aspects, the transformed cell is a cell suitable for production of AAV vectors as described herein.

Another aspect relates to a transgenic animal comprising a polynucleotide, an expression cassette, a vector, and/or the transformed cell described herein. In some aspects, a transgenic animal is a laboratory animal, e.g., a mouse rat, dog, or monkey. In some aspects, the animal is a model of a disease.

### Pharmaceutical Composition

The present disclosure provides compositions comprising any of the isolated polynucleotides, rAAV vectors, and/or rAAV viral vectors described herein. In some aspects, the compositions can be pharmaceutical compositions. Accordingly, the present disclosure provides pharmaceutical compositions comprising any of the isolated polynucleotides, rAAV vectors, and/or rAAV viral vectors described herein.

By “pharmaceutically acceptable” it is meant the carrier, diluent or excipient must be compatible with the other ingredients of the formulation and not deleterious to the recipient thereof. Examples of carriers include, but are not limited to, liposomes, nanoparticles, ointments, micelles, microspheres, microparticles, creams, emulsions, and gels. Examples of excipients include, but are not limited to, anti-adherents such as magnesium stearate, binders such as saccharides and their derivatives (sucrose, lactose, starches, cellulose, sugar alcohols and the like) proteins like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluents include, but are not limited to, water, alcohol, saline solution, glycol, mineral oil and dimethyl sulfoxide (DMSO). “Pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15^{th} Ed. (Mack Publ. Co., Easton).

Pharmaceutically acceptable carriers, excipients, and stabilizers are well known in the art, for example Remington's The Science and Practice of Pharmacy, 23^{rd} edition, Osol, A. Ed. (2020). Other examples of pharmaceutically acceptable carriers, excipients, or stabilizers that are nontoxic to recipients at the dosages and concentrations employed, may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

The pharmaceutical composition, as described herein, may be formulated by any methods known or developed in the art of pharmacology, which include but are not limited to contacting the active ingredients (e.g., viral particles or recombinant vectors) with an excipient and/or additive and/or other accessory ingredient, dividing or packaging the product to a dose unit. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition may further comprise saline, lipidoids, liposomes, lipid nanoparticles, polymers, lipoplexes, core-shell nanoparticles, peptides, proteins, cells transfected with viral vectors (e.g., for transplantation into a subject), nanoparticle mimics or combinations thereof. In some aspects, the pharmaceutical composition is formulated as a nanoparticle. In some aspects, the nanoparticle is a self-assembled nucleic acid nanoparticle.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations can include one or more excipients and/or additives, each in an amount that together increases the stability of the viral vector, increases cell transfection or transduction by the viral vector, increases the expression of viral vector encoded protein, and/or alters the release profile of viral vector encoded proteins. In some aspects, the pharmaceutical composition comprises an excipient and/or additive. Non limiting examples of excipients and/or additives include solvents, dispersion media, diluents, or other liquid vehicles, dispersion or suspension aids, surface active agents, isotonic agents, thickening or emulsifying agents, preservatives, or combination thereof.

In some aspects, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffinose and/or mannitol.

In some aspects, a pharmaceutical composition of the present disclosure can comprise phosphate-buffered saline (PBS), D-sorbitol or any combination thereof.

In some aspects, a pharmaceutical composition can comprise PBS, wherein the PBS is present at a concentration of about 100 mM to about 500 mM, or about 200 mM to about 400 mM, or about 300 mM to about 400 mM. In some aspects, the sodium chloride can be present at a concentration of about 350 mM.

In some aspects, a pharmaceutical composition can comprise D-sorbitol, wherein the D-sorbitol is present at a concentration of about 1% to about 10%, or about 2.5% to about 7.5%. In some aspects, the D-sorbitol can be present at a concentration of about 5%.

Thus, the present disclosure provides a pharmaceutical composition comprising an rAAV vector and/or rAAV viral vector of the present disclosure in a 350 mM phosphate-buffered saline solution comprising D-sorbitol at a concentration of 5%.

### Methods of Use

Provided herein are methods for delivering an AP4M1 polynucleotide to a cell, a tissue, an organ, an animal or a subject to increase production of AP4M1, e.g., for therapeutic or research purposes in vitro, ex vivo, or in vivo, using the compositions and pharmaceutical compositions, e.g., administering or contacting the cell, tissue, organ, animal, or subject with a therapeutic effective amount of the composition or pharmaceutical composition. In one aspect, the subject is a mammal. The subject can be human. Thus, one aspect relates to a method of expressing an AP4M1 polynucleotide in a cell, comprising contacting the cell with the polynucleotide, the expression cassette, the vector, the rAAV vector, and/or the pharmaceutical composition described herein, thereby expressing the AP4M1 polynucleotide in the cell. In some aspects, the cell is an in vitro cell, an ex vivo cell, or an in vivo cell.

The cell(s) into which the vector (e.g., viral or non-viral vector) can be introduced can be of any type, including but not limited to neural cells (including cells of the peripheral and central nervous systems, in particular, brain cells such as neurons, oligodendrocytes, glial cells, astrocytes), lung cells, cells of the eye (including retinal cells, retinal pigment epithelium, and corneal cells), epithelial cells (e.g., gut and respiratory epithelial cells), skeletal muscle cells (including myoblasts, myotubes and myofibers), diaphragm muscle cells, dendritic cells, pancreatic cells (including islet cells), hepatic cells, a cell of the gastrointestinal tract (including smooth muscle cells, epithelial cells), heart cells (including cardiomyocytes), bone cells (e.g., bone marrow stem cells), hematopoietic stem cells, spleen cells, keratinocytes, fibroblasts, endothelial cells, prostate cells, joint cells (including, e.g., cartilage, meniscus, synovium and bone marrow), germ cells, and the like. Alternatively, the cell may be any progenitor cell. As a further alternative, the cell can be a stem cell (e.g., neural stem cell, liver stem cell). As still a further alternative, the cell may be a cancer or tumor cell (cancers and tumors are described above). Moreover, the cells can be from any species of origin, as indicated above.

Another aspect relates to a method of expressing an AP4M1 polynucleotide in a subject, comprising delivering to the subject the polynucleotide, the expression cassette, the vector, the rAAV vector and/or the pharmaceutical composition described herein, thereby expressing the AP4M1 polynucleotide in the subject. In some aspects, the subject is an animal model of SPG50, or other disorder associated with aberrantAP4M1 gene expression.

An additional aspect relates to a method of treating a disorder associated with aberrant expression of an AP4M1 gene or aberrant activity of an AP4M1 gene product in a subject in need thereof, comprising delivering to the subject a therapeutically effective amount of the polynucleotide, the expression cassette, the vector, the rAAV vector and/or the pharmaceutical composition described herein, thereby treating the disorder associated with aberrant expression of the AP4M1 gene in the subject. In some aspects, the disorder associated with expression of the AP4M1 gene is hereditary spastic paraplegia, such as SPG50.

The term “disorder associated with aberrant expression of an AP4M1 gene” as used herein refers to a disease, disorder, syndrome, or condition that is caused by or a symptom of decreased or altered expression of the AP4M1 gene in a subject relative to the expression level or activity in a normal subject or in a population. In some aspects, the disease and/or disorder can be a genetic disorder involving the AP4M1 gene. A genetic disorder involving the AP4M1 gene can be AP4M1 loss, misfunction and/or deficiency. Genetic disorders involving the AP4M1 gene include, but are not limited to, spastic paraplegia type 50 (SPG50).

In some aspects, the disease can be a disorder involving the AP4M1 protein. A genetic disorder involving an AP4M1 protein can be AP4M1 loss, misfunction and/or deficiency.

In some aspects, a disease can be a disease that is characterized by the loss-of-function of at least one copy of the AP4M1 gene in the genome of a subject. In some aspects, a disease can be a disease that is characterized by a decrease in function of at least one copy of the AP4M1 gene in the genome of a subject. In some aspects, a disease can be a disease that is characterized by at least one mutation in at least one mutation in at least one copy of the AP4M1 gene in the genome of the subject.

A subject in the methods provided herein can be deficient in AP4M1. As used herein, “AP4M1 deficiency” means that a subject can have one or more mutations in the AP4M1 gene or lacks a functional AP4M1 gene. As used herein, “AP4M1 deficiency” means that a subject can have one or more mutations in the AP4M1 gene or lacks a functional AP4M1 protein.

A mutation in an AP4M1 gene or AP4M1 protein can be any type of mutation that is known in the art. Non-limiting examples of mutations include somatic mutations, single nucleotide variants (SNVs), nonsense mutations, insertions, deletions, duplications, frameshift mutations, repeat expansions, short insertions and deletions (INDELs), long INDELs, alternative splicing, the products of alternative splicing, altered initiation of translation, the products of altered initiation of translation, proteomic cleavage, the products of proteomic cleavage.

In some aspects, a disease can be a disease that is characterized by a decrease in expression of the AP4M1 gene in a subject as compared to a control subject that does not have the disease. In some aspects, the decrease in expression can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

In some aspects, a disease can be a disease that is characterized by a decrease in the amount of AP4M1 protein in a subject as compared to a control subject that does not have the disease. In some aspects, the decrease in the amount of AP4M1 protein can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

In some aspects, a disease can be a disease that is characterized by a decrease in the activity of AP4M1 protein in a subject as compared to a control subject that does not have the disease. In some aspects, the decrease in the activity of AP4M1 protein can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

Another aspect relates to a method of treating spastic paraplegia type 50 (SPG50) in a subject comprising delivering to the subject a therapeutically effective amount of the polynucleotide, the expression cassette, the vector, the rAAV vector and/or the pharmaceutical composition described herein, thereby treating SPG50 in the subject. In some aspects, the subject is an animal model of SPG50, or other disorder associated with aberrantAP4M1 gene expression.

The term “subject” as used herein refers to any individual or patient to which the subject methods are performed. The terms “subject” and “patient” are used interchangeably herein. Generally, the subject is human, although as will be appreciated by those in the art, the subject may be an animal. In some aspects, the subject is a human. In some aspects, the subject is a human child, e.g., a child of less than five years of age. In some aspects, the subject is a human newborn, e.g., a newborn of less than one month, less than two months, less than three months, or less than four months of age. In some aspects, a subject can be less than 0.5 years of age, or less than 1 year of age, or less than 1.5 years of age, or less than 2 years of age, or at less than 2.5 years of age, or less than 3 years of age, or less than 3.5 years of age, or less than 3.5 years of age, or less than 4 years of age, or less than 4.5 years of age, or less than 5 years of age, or less than 5.5 years of age, or less than 6 years of age, or less than 6.5 years of age, or less than 7 years of age, or less than 7.5 years of age, or less than 8 years of age, or less than 8.5 years of age, or less than 9 years of age, or less than 9.5 years of age, or less than 10 years of age. In some aspects the subject can be less than 11 years of age, less than 12 years of age, less than 13 years of age, less than 14 years of age, less than 15 years of age, less than 20 years of age, less than 30 years of age, less than 40 years of age, less than 50 years of age, less than 60 years of age, less than 70 years of age, less than 80 years of age, less than 90 years of age, less than 100 years of age, less than 110 years of age, or less than 120 years of age. In some aspects, a subject can be less than 0.5 years of age. In some aspects, a subject can be less than 4 years of age. In some aspects, a subject can be less than 10 years of age. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject. A subject to be treated using the methods, compositions, pharmaceutical compositions, rAAV vectors or rAAV viral vectors of the present disclosure can have any of the diseases and/or symptoms described herein.

The term “treatment” is used interchangeably herein with the term “therapeutic method” and refers to therapeutic treatments or measures that cure, slow down, lessen symptoms of, and/or halt progression of a diagnosed pathologic conditions or disorder. “Prophylactic treatment” refers to prophylactic/preventative measures. Those in need of treatment may include individuals already having a particular medical disorder as well as those who may ultimately acquire the disorder (i.e., those needing preventive measures). As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein, “preventing” or “prevention” of a disease refers to preventing the symptoms or disease from occurring in a subject that is predisposed or does not yet display symptoms of the disease.

Methods of treatment can alleviate one or more symptoms of a disease and/or disorder described herein. In an aspect, delivery of compositions described herein can prevent or delay development of detectable symptoms, if administered to a subject carrying a mutation in the AP4M1 gene before symptoms become detectable. Therefore, treatment can be therapeutic or prophylactic. Therapy refers to inhibition or reversal of established symptoms or phenotype. Therapy can also mean delay of onset of symptoms or phenotype. Prophylaxis means inhibiting or preventing development of symptoms in subjects not already displaying overt symptoms. Subjects not displaying overt symptoms can be identified early in life as carrying a loss of function mutation in the AP4M1 gene by appropriate genetic testing performed before 18 months, 12 months, or 6 months of age.

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of a SPG50 disease). In some aspects, the effective amount will depend on the size and nature of the application in question. It will also depend on the nature and sensitivity of the target subject and the methods in use. The skilled artisan will be able to determine the effective amount based on these and other considerations. The effective amount may comprise, consist essentially of, or consist of one or more administrations of a composition depending on the embodiment. Dosages of the virus vectors to be administered to a subject will depend upon the mode of administration, the disease or condition to be treated, the individual subject's condition, the particular virus vector, and the nucleic acid to be delivered, and can be determined in a routine manner. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 109 vector genomes to as much as 10¹⁷ vector genomes per administration.

In some aspects of the methods described herein, the number of viral particles (e.g., rAAV viral vectors) administered to the subject ranges from about 109 to about 10¹⁷. In some aspects, about 10¹⁰ to about 10¹², about 10¹¹ to about 10¹³, about 10¹¹ to about 10¹², about 10¹¹ to about 10¹⁴, about 10¹² to about 10¹⁶, about 10¹³ to about 10¹⁶, about 10¹⁴ to about 10¹⁵, about 5×10¹¹ to about 5×10¹², about 10¹¹ to about 10¹⁸, about 10¹³ to about 10¹⁶, or about 10¹² to about 10¹³ viral particles are administered to the subject.

In some aspects of the methods described herein, the number of viral particles (e.g., rAAV viral vectors) administered to the subject is at least about 10¹⁰, or at least about 10¹¹, or at least about 10¹², or at least about 10¹³, or at least about 10¹⁴, or at least about 10¹¹, or at least about 10¹⁶, or at least about 10¹⁷ viral particles.

In some aspects of the methods described herein, the number of viral particles (e.g., rAAV viral vectors) administered to the subject is ranging from about 10¹¹ to about 10¹⁸ viral vector particles. In other aspects of the methods described herein, the number of viral particles (e.g., rAAV viral vectors) administered to the subject is ranging from about 10¹³ to about 10¹⁶ viral vector particles. In some aspects of the methods described herein, the number of viral particles (e.g., rAAV viral vectors) administered to the subject is about 1.25E11, 2.5E11 or 5E11 vector particles.

In some aspects of the methods described herein, the number of viral particles (e.g., rAAV viral vectors) administered to the subject can depend on the age of the subject. In non-limiting examples, a subject that is 7 years of age or older can be administered about 10×10¹⁴ viral particles, a subject that is about 4 years of age to about 7 years of age can be administered about 10×10¹⁴ viral particles, a subject that is about 3 years of age to about 4 years of age can be administered about 9×10¹⁴ viral particles, a subject that is about 2 years of age to about 3 years of age can be about 8.2×10¹⁴ viral particles, a subject that is about 1 year of age to about 2 years of age can be administered about 7.3×10¹⁴ viral particles, a subject that is about 0.5 years of age to about 1 year of age can be administered about 4×10¹⁴ viral particles, or a subject that is less than 0.5 years of age can be administered 3×10¹⁴ viral particles.

In some aspects, the amounts of viral particles in a composition, pharmaceutical composition, or the amount of viral particles administered to a patient can calculated based on the percentage of viral particles that are predicted to contain viral genomes.

The terms “delivering” means providing or administering a pharmaceutical composition in a therapeutically effective amount to the subject in need of treatment. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian. As such, administration routes include but are not limited to intravenously, intrathecally (IT), intracisternal-magna (ICM),intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally; such introduction may also be intra-arterial, intracardiac, subventricular, epidural, intracerebral, intracerebroventricular, sub-retinal, intravitreal, intraarticular, intraperitoneal, intrauterine, intranerve or any combination thereof. In some aspects, the viral particles are delivered to a desired target tissue, e.g., to the lung, eye, or CNS, as non-limiting examples. In some aspects, delivery of viral particles is systemic. The intracisternal route of administration involves administration of a drug directly into the cerebrospinal fluid of the brain ventricles. It could be performed by direct injection into the cisterna magna or via a permanently positioned tube. In some aspects, the rAAV viral vectors of the present disclosure are administered intrathecally (IT). In some aspects, the rAAV viral vectors of the present disclosure are administered lumbar intrathecally.

Administration of the rAAV vectors, rAAV viral vectors, compositions or pharmaceutical compositions of this disclosure can be effected in one dose, continuously or intermittently throughout the course of treatment. In some aspects, the rAAV vectors, rAAV viral vectors, compositions, or pharmaceutical compositions of this disclosure are parenterally administered by injection, infusion, or implantation.

In some aspects, the subject is administered one single dose of a recombinant rAAV provided herein in its lifetime. In some aspects, the subject is administered repeat doses of the recombinant rAAV provided herein. The form or serotype of rAAV can differ in subsequent doses versus the initial dose. These repeat doses may contain the same amount of rAAV particles or they may contain different amounts of rAAV particles. In some aspects, the subject is administered repeat doses of the rAAV about every 6 months, about every 9 months, about every 12 months, about every 15 months, about every 18 months, about every 2 years, about every 3 years, about every 4 years, about every 5 years, about every 6 years, about every 7 years, about every 8 years, about every 9 years, or about every 10 years.

In one aspect, a method of treating spastic paraplegia type 50 (SPG50) in a subject comprises administering to the subject a therapeutically effective amount of the polynucleotide, the expression cassette the vector, the rAAV vector and/or the pharmaceutical composition described herein, thereby treating SPG50 in the subject. In another aspect, administering to the subject a therapeutically effective amount of the polynucleotide, the expression cassette, the vector, and/or the rAAV vector described herein comprises administering a pharmaceutical composition comprising a therapeutically effective amount of the polynucleotide, expression cassette, vector, and/or rAAV vector and a pharmaceutically acceptable carrier.

The methods of treatment and prevention disclosed herein may be combined with appropriate diagnostic techniques to identify and select patients for the therapy or prevention.

The disclosure provides methods of increasing the level of a protein in a host cell, comprising contacting the host cell with any one of the rAAV viral vectors disclosed herein, wherein the rAAV viral vectors comprises any one of the rAAV vectors disclosed herein, comprising a transgene nucleic acid molecule encoding the protein. In some aspects, the protein is a therapeutic protein. In some aspects, the host cell is in vitro, in vivo, or ex vivo. In some aspects, the host cell is derived from a subject. In some aspects, the subject suffers from a disorder, which results in a reduced level and/or functionality of the protein, as compared to the level and/or functionality of the protein in a normal subject.

In some aspects, the level of the protein is increased to level of about 1×10-7 ng, about 3×10-7 ng, about 5×10-7 ng, about 7×10-7 ng, about 9×10-7 ng, about 1×10-6 ng, about 2×10-6 ng, about 3×10-6 ng, about 4×10-6 ng, about 6×10-6 ng, about 7×10-6 ng, about 8×10-6 ng, about 9×10-6 ng, about 10×10-6 ng, about 12×10-6 ng, about 14×10-6 ng, about 16×10-6 ng, about 18×10-6 ng, about 20×10-6 ng, about 25×10-6 ng, about 30×10-6 ng, about 35×10-6 ng, about 40×10-6 ng, about 45×10-6 ng, about 50×10-6 ng, about 55×10-6 ng, about 60×10-6 ng, about 65×10-6 ng, about 70×10-6 ng, about 75×10-6 ng, about 80×10-6 ng, about 85×10-6 ng, about 90×10-6 ng, about 95×10-6 ng, about 10×10-5 ng, about 20×10-5 ng, about 30×10-5 ng, about 40×10-5 ng, about 50×10-5 ng, about 60×10-5 ng, about 70×10-5 ng, about 80×10-5 ng, or about 90×10-5 ng in the host cell.

The expression levels of a gene (e.g., AP4M1) or a protein (e.g., AP4M1) may be determined by any suitable method known in the art or described herein. Protein levels may be determined, for example, by western Blotting, immunohistochemistry and flow cytometry. Gene expression may be determined, for example, by quantitative PCR, gene sequencing, and RNA sequencing.

The disclosure provides methods of introducing a gene of interest to a cell in a subject comprising contacting the cell with an effective amount of any one of the rAAV viral vectors disclosed herein, wherein the rAAV viral vectors contain any one of the rAAV vectors disclosed herein, comprising the gene of interest.

In some aspects, the rAAV viral vectors of the present disclosure repair a gene deficiency in a subject. In some aspects, the ratio of repaired target polynucleotide or polypeptide to unrepaired target polynucleotide or polypeptide in a successfully treated cell, tissue, organ or subject is at least about 1.5:1, about 2:1, about 3:1, about 4:1, about 5:1, about 6:1, about 7:1, about 8:1, about 9:1, about 10:1, about 20:1, about 50:1, about 100:1, about 1000:1, about 10,000:1, about 100,000:1, or about 1,000,000:1. The amount or ratio of repaired target polynucleotide or polypeptide can be determined by any method known in the art, including but not limited to western blot, northern blot, Southern blot, PCR, sequencing, mass spectrometry, flow cytometry, immunohistochemistry, immunofluorescence, fluorescence in situ hybridization, next generation sequencing, immunoblot, and ELISA.

In certain aspects, a polynucleotide, expression cassette, vector, the rAAV vector, the pharmaceutical composition and/or transformed cell is delivered to the nervous system of the subject, e.g., directly to the nervous system of the subject. In some aspects, the polynucleotide, expression cassette, vector, the rAAV vector, the pharmaceutical composition and/or transformed cell is delivered by intrathecal, intranerve, intracerebral, intraventricular, intranasal, intra-aural, intra-ocular, or peri-ocular delivery, or any combination thereof. In some aspects, the polynucleotide, expression cassette, vector, the rAAV vector, the pharmaceutical composition and/or transformed cell is delivered intravenously. In another aspect, the polynucleotide, expression cassette, the rAAV vector, the pharmaceutical composition and/or vector is delivered by injection, electroporation, gene gun, sonoporation, magnetofection, hydrodynamic delivery, or other physical or chemical methods. In some aspects, the viral vector is administered to the CNS, the peripheral nervous system, or both.

In some aspects of the methods of the present disclosure, a subject can also be administered a prophylactic immunosuppressant treatment regimen in addition to being administered an rAAV vector or rAAV viral vector of the present disclosure. In some aspects, an immunosuppressant treatment regimen can comprise administering at least one immunosuppressive therapeutic. Non limiting examples of immunosuppressive therapeutics include, but are not limited to, Sirolimus (rapamycin), acetaminophen, diphenhydramine, IV methylprednisolone, prednisone, or any combination thereof. An immunosuppressive therapeutic can be administered prior to the day of administration of the rAAV vector and/or rAAV viral vector, on the same day as the administration of the rAAV vector and/or rAAV viral vector, or any day following the administration of the rAAV vector and/or rAAV viral vector.

In certain aspects, a polynucleotide, expression cassette, vector, rAAV vector or pharmaceutical composition is administered to a subject in need thereof as early as possible in the life of the subject, e.g., as soon as the subject is diagnosed with aberrant AP4M1expression and/or aberrant AP4M1activity. For example, a polynucleotide, expression cassette, vector, rAAV vector or pharmaceutical composition can be administered to a subjected that is 10 years-old or less. The subject can be 10 years-old, 9 years-old, 4 years-old, 3 years-old, 2 years-old, 1-year-old, 9 months-old, 6 months-old, 3 months-old, 1-month-old or less. In some aspects, the polynucleotide is administered to a newborn subject, e.g., after newborn screening has identified aberrant AP4M1 expression and/or aberrant AP4M1 activity. In some aspects, the polynucleotide is administered to a fetus in utero, e.g., after prenatal screening has identified aberrant AP4M1 expression and/or aberrant AP4M1 activity. In some aspects, the polynucleotide is administered to a subject as soon as the subject develops symptoms associated with aberrant AP4M1 expression and/or aberrant AP4M1 activity or is suspected or diagnosed as having aberrant AP4M1 expression and/or aberrant AP4M1 activity.

In some aspects, a polynucleotide, expression cassette, vector, rAAV vector or pharmaceutical composition is administered to a subject before the subject develops symptoms associated with aberrant AP4M1 expression and/or aberrant AP4M1 activity, e.g., a subject that is suspected or diagnosed as having aberrant AP4M1 expression and/or aberrant AP4M1 activity but has not started to exhibit symptoms.

In one aspect, the method of treating spastic paraplegia type 50 (SPG50) in a subject comprises administering to the subject a therapeutically effective amount of a polynucleotide, an expression cassette, a vector, arAAV vector and/or a pharmaceutical composition described herein, thereby treating SPG50 in the subject. In another aspect, administering to the subject a therapeutically effective amount of a polynucleotide, an expression cassette, a vector, and/or a rAAV vector comprises administering a pharmaceutical composition comprising a therapeutically effective amount of the polynucleotide, expression cassette, and/or vector and a pharmaceutically acceptable carrier. In one aspect, administering a therapeutically effective amount of a polynucleotide, an expression cassette, a vector, a rAAV vector and/or a pharmaceutical composition to a subject can reduce or alleviate one or more symptoms of SPG50. For example, administering a therapeutically effective amount of a polynucleotide, an expression cassette, a vector, a rAAV vector and/or a pharmaceutical composition to a subject reduce or alleviate SPG50 symptoms such as low muscle tone, spasticity, paralysis in the lower limbs, dystonia, ataxia, or seizures. In some aspects, administering the polynucleotide, expression cassette, vector, rAAV vector or pharmaceutical composition described herein can improve motor coordination and/or grip strength in the subject.

In some aspects, administering the polynucleotide, expression cassette, vector, rAAV vector or pharmaceutical composition can increase AP4M1 levels in the brain of the subject. For example, AP4M1 levels can be increased in the cortex, subcortex, brain stem and/or cerebellum of the subject.

In some aspects, administering the polynucleotide, expression cassette, vector, rAAV vector or pharmaceutical composition can induce no immune response or a minimal immune response from the subject. As described in the example below, the administration of the polynucleotide, expression cassette, vector, rAAV vector or pharmaceutical composition described herein may induce an immune response in the subject. Immune response can for example be assessed by measuring the level of interferon gamma (INFγ) in the subject. In various aspects, administering the polynucleotide, expression cassette, vector, rAAV vector or pharmaceutical composition does not induce an immune response (i.e., does not induce an increase in INFγ secretion and/or production in the subject). In other aspects, administering the polynucleotide, expression cassette, vector, rAAV vector or pharmaceutical composition induces a minimal immune response (i.e., induces a minimal or not significative increase in INFγ secretion and/or production in the subject).

In some aspects, administering the polynucleotide, expression cassette, vector, rAAV vector or pharmaceutical composition can induce no serum toxicity or a minimal serum toxicity in the subject. Serum toxicity can for example be assessed by measuring the levels of aspartate transaminase (AST), total bilirubin (TBIL), albumin (ALB), creatine kinase (CK), and blood urea nitrogen (BUN) in the serum of the subject. In various aspects, administering the polynucleotide, expression cassette, vector, rAAV vector or pharmaceutical composition does not induce serum toxicity (i.e., does not induce an increase in AST, TBIL, ALB, CK, and/or BUN). In other aspects, administering the polynucleotide, expression cassette, vector, rAAV vector or pharmaceutical composition induces a minimal serum toxicity (i.e., induces a minimal or not significative increase in the levels of AST, TBIL, ALB, CK, and/or BUN).

### Methods of Manufacture

A variety of approaches may be used to produce rAAV viral vectors of the present disclosure. In some aspects, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In some aspects, the cell is a packaging or helper cell line. In some aspects, the helper cell line is eukaryotic cell; for example, an HEK 293 cell or 293T cell. In some aspects, the helper cell is a yeast cell or an insect cell.

In some aspects, the cell comprises a nucleic acid encoding a tetracycline activator protein; and a promoter that regulates expression of the tetracycline activator protein. In some aspects, the promoter that regulates expression of the tetracycline activator protein is a constitutive promoter. In some aspects, the promoter is a phosphoglycerate kinase promoter (PGK) or a CMV promoter.

A helper plasmid can comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are known in the art, see, e.g., U.S. Patent Pub. No. 2004/0235174 A1, incorporated herein by reference. As stated therein, an AAV helper plasmid may contain as helper virus DNA sequences, by way of non-limiting example, the Ad5 genes E2A, E4 and VA, controlled by their respective original promoters or by heterologous promoters. AAV helper plasmids may additionally contain an expression cassette for the expression of a marker protein such as a fluorescent protein to permit the simple detection of transfection of a desired target cell.

The disclosure provides methods of producing rAAV viral vectors comprising transfecting a packaging cell line with any one of the AAV helper plasmids disclosed herein; and any one of the rAAV vectors disclosed herein. In some aspects, the AAV helper plasmid and rAAV vector are co-transfected into the packaging cell line. In some aspects, the cell line is a mammalian cell line, for example, human embryonic kidney (HEK) 293 cell line. The disclosure provides cells comprising any one of the rAAV vectors and/or rAAV viral vectors disclosed herein.

As used herein, the term “helper” in reference to a virus or plasmid refers to a virus or plasmid used to provide the additional components necessary for replication and packaging of any one of the rAAV vectors disclosed herein. The components encoded by a helper virus may include any genes required for virion assembly, encapsidation, genome replication, and/or packaging. For example, the helper virus or plasmid may encode necessary enzymes for the replication of the viral genome. Non-limiting examples of helper viruses and plasmids suitable for use with AAV constructs include pHELP (plasmid), adenovirus (virus), or herpesvirus (virus). In some aspects, the pHELP plasmid may be the pHELPK plasmid, wherein the ampicillin expression cassette is exchanged with a kanamycin expression cassette.

As used herein, a packaging cell (or a helper cell) is a cell used to produce viral vectors. Producing recombinant AAV viral vectors requires Rep and Cap proteins provided in trans as well as gene sequences from Adenovirus that help AAV replicate. In some aspects, Packaging/helper cells contain a plasmid is stably incorporated into the genome of the cell. In other aspects, the packaging cell may be transiently transfected. Typically, a packaging cell is a eukaryotic cell, such as a mammalian cell or an insect cell.

### Kits

The isolated polynucleotides, rAAV vectors, rAAV viral vectors, compositions, and/or pharmaceutical compositions described herein may be assembled into pharmaceutical or diagnostic or research kits to facilitate their use in therapeutic, diagnostic, or research applications. In some aspects, the kits of the present disclosure include any one of the isolated polynucleotides, rAAV vectors, rAAV viral vectors, compositions, pharmaceutical compositions, host cells, isolated tissues, as described herein.

In some aspects, a kit further comprises instructions for use. Specifically, such kits may include one or more agents described herein, along with instructions describing the intended application and the proper use of these agents. In some aspects, the kit may include instructions for mixing one or more components of the kit and/or isolating and mixing a sample and applying to a subject. In some aspects, agents in a kit are in a pharmaceutical formulation and dosage suitable for a particular application and for a method of administration of the agents. Kits for research purposes can contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In some aspects, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In some aspects, the preservation solution contains an amount of metalloprotease inhibitors.

In some aspects, the kit contains any one or more of the components described herein in one or more containers. Thus, in some aspects, the kit may include a container housing agents described herein. The agents may be in the form of a liquid, gel or solid (powder). The agents may be prepared sterilely, packaged in a syringe and shipped refrigerated. Alternatively, they may be housed in a vial or other container for storage. A second container can have other agents prepared sterilely. Alternatively, the kit can include the active agents premixed and shipped in a syringe, vial, tube, or other container. The kit can have one or more or all of the components required to administer the agents to a subject, such as a syringe, topical application devices, or IV needle tubing and bag.

### Further Definitions

Unless the context indicates otherwise, it is specifically intended that the various features described herein can be used in any combination. Moreover, the disclosure also contemplates that, in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified aspects, embodiments, features, and terms intend to include both the recited aspect, embodiment, feature, or term and biological equivalents thereof.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (RI. Freshney, ed. (1987)).

All numerical designations, e.g., pH, temperature, time, concentration, and molecular weight, including ranges, are approximations which are varied (+) or (−) by increments of 1.0 or 0.1, as appropriate, or, alternatively, by a variation of +/−15%, 10%, 5%, 2%. It is to be understood, although not always explicitly stated, that all numerical designations are preceded by the term “about”. It also is to be understood, although not always explicitly stated, that the reagents described herein are merely exemplary and that equivalents of such are known in the art. The term “about,” as used herein when referring to a measurable value such as an amount or concentration and the like, is meant to encompass variations of 20%, 10%, 5%, 1%, 0.5%, or even 0.1% of the specified amount.

The terms “acceptable” or “effective,” when used to describe the selection of any components, ranges, dose forms, etc. disclosed herein intend that said component, range, dose form, etc. is suitable for the disclosed purpose.

Also, as used herein, “and/or” refers to and encompasses any and all possible combinations of one or more of the associated listed items, as well as the lack of combinations when interpreted in the alternative (“or”).

Unless specifically recited, the term “host cell” includes a eukaryotic host cell, including, for example, fungal cells, yeast cells, higher plant cells, insect cells and mammalian cells. Non-limiting examples of eukaryotic host cells include simian, bovine, porcine, murine, rat, avian, reptilian and human, e.g., HEK293 cells and 293T cells.

The term “isolated” as used herein refers to molecules or biologicals or cellular materials being substantially free from other materials.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, consisting essentially of, or consisting of purine and pyrimidine bases or other natural, chemically or biochemically modified, non-natural, or derivatized nucleotide bases.

A “gene” refers to a polynucleotide containing at least one open reading frame (ORF) that is capable of encoding a particular polypeptide or protein. A “gene product” or, alternatively, a “gene expression product” refers to the amino acid sequence (e.g., peptide or polypeptide) generated when a gene is transcribed and translated.

As used herein, “expression” refers to the two-step process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell.

“Under transcriptional control” is a term well understood in the art and indicates that transcription of a polynucleotide sequence, usually a DNA sequence, depends on its being operatively linked to an element that contributes to the initiation of, or promotes, transcription. “Operatively linked” intends that the polynucleotides are arranged in a manner that allows them to function in a cell. In one aspect, promoters can be operatively linked to the downstream sequences.

The term “encode” as it is applied to polynucleotides and/or nucleic acid sequences refers to a polynucleotide and/or nucleic acid sequence which is said to “encode” a polypeptide if its base sequence is identical to the base sequence of the RNA transcript (e.g., mRNA transcript) that is translated into the polypeptide and/or a fragment thereof. The antisense strand is the complement of such a nucleic acid, and the encoding sequence can be deduced therefrom.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise, consist essentially of, or consist of a protein's or peptide's sequence. As used herein the term “amino acid” refers to either natural and/or unnatural or synthetic amino acids, including glycine and both the D and L optical isomers, amino acid analogs and peptidomimetics.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g., across a cell membrane, into a cell membrane, or into the nucleus. In some aspects, the signal peptide is removed following localization. Examples of signal peptides are well known in the art. Non-limiting examples are those described in U.S. Pat. Nos. 8,853,381, 5,958,736, and 8,795,965. In some aspects, the signal peptide can be an IDUA signal peptide.

The terms “equivalent” or “biological equivalent” are used interchangeably when referring to a particular molecule, biological material, or cellular material and intend those having minimal homology while still maintaining desired structure or functionality. Non-limiting examples of equivalent polypeptides include a polypeptide having at least about 60%, at least about 65%, at least about 70%, at least about 75%, at least about 80%, at least about 85%, at least about 90%, at least about 95% identity or at least about 99% identity to a reference polypeptide (for instance, a wild-type polypeptide); or a polypeptide which is encoded by a polynucleotide having at least about 70%, at least about 75%, at least about 80%, at least about 85%, at least about 90%, at least about 95% identity, at least about 97% sequence identity or at least about 99% sequence identity to the reference polynucleotide (for instance, a wild-type polynucleotide).

“Homology” or “identity” or “similarity” refers to sequence similarity between two peptides or between two nucleic acid molecules. Percent identity can be determined by comparing a position in each sequence that may be aligned for purposes of comparison. When a position in the compared sequence is occupied by the same base or amino acid, then the molecules are identical at that position. A degree of identity between sequences is a function of the number of matching positions shared by the sequences. “Unrelated” or “non-homologous” sequences share less than 40% identity, less than 25% identity, with one of the sequences of the present disclosure. Alignment and percent sequence identity may be determined for the nucleic acid or amino acid sequences provided herein by importing said nucleic acid or amino acid sequences into and using ClustalW (available at genome.jp/tools-bin/clustalw/). For example, the ClustalW parameters used for performing the protein sequence alignments found herein were generated using the Gonnet (for protein) weight matrix. In some aspects, the ClustalW parameters used for performing nucleic acid sequence alignments using the nucleic acid sequences found herein are generated using the ClustalW (for DNA) weight matrix.

As used herein, amino acid modifications may be amino acid substitutions, amino acid deletions or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g., charge, hydrophobicity or size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one charged or polar residue for another, such as the substitution of arginine for lysine, glutamic acid for aspartic acid, glutamine for asparagine, and the like. Other illustrative examples of conservative substitutions include the changes of: alanine to serine; asparagine to glutamine or histidine; aspartate to glutamate; cysteine to serine; glycine to proline; histidine to asparagine or glutamine; lysine to arginine, glutamine, or glutamate; phenylalanine to tyrosine, serine to threonine; threonine to serine; tryptophan to tyrosine; tyrosine to tryptophan or phenylalanine; and the like.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is a DNA molecule that is typically separate from and capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or, alternatively, the proteins produced may act as toxins under similar circumstances. It is known in the art that while plasmid vectors often exist as extrachromosomal circular DNA molecules, plasmid vectors may also be designed to be stably integrated into a host chromosome either randomly or in a targeted manner, and such integration may be accomplished using either a circular plasmid or a plasmid that has been linearized prior to introduction into the host cell.

“Plasmids” used in genetic engineering are called “plasmid vectors”. Many plasmids are commercially available for such uses. The gene to be replicated is inserted into copies of a plasmid containing genes that make cells resistant to particular antibiotics, and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria or eukaryotic cells containing a plasmid harboring the gene of interest, which can be induced to produce large amounts of proteins from the inserted gene.

In aspects where gene transfer is mediated by a DNA viral vector, such as an adenovirus (Ad) or adeno-associated virus (AAV), a vector construct refers to the polynucleotide comprising, consisting essentially of, or consisting of the viral genome or part thereof, and a transgene.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected), or a group of tissues making up an organ or part or region of the body of an organism. The tissue may comprise, consist essentially of, or consist of a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

Any single term, single element, single phrase, group of terms, group of phrases, or group of elements described herein can be each be specifically excluded from the claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects are described in terms of Markush groups or other grouping of alternatives, those skilled in the art will recognize that the features and aspects are also thereby described in terms of any individual member or subgroup of members of the Markush group or other group.

The following are provided for exemplification purposes only and are not intended to limit the scope of the embodiments described in broad terms above.

### EXAMPLES

### Example 1: Experimental Design

Adaptor protein (AP) complexes select transmembrane proteins as cargos for inclusion, recruit coat proteins to generate vesicles, and transport the cargos to different part of the cells. As one of the five members in the AP complex family, AP-4 complex assembles as a hetero-tetramers (β4, ε, μ4, and σ4) and requires the presence of all subunits to maintain functionality. Loss-of-function mutations in any of the genes (AP4B1, AP4E1, AP4M1, and AP4S1) cause a severe recessive neurological disorder with early-onset progressive spastic paraplegia (SPG) and intellectual disability. Recent reports uncovered that Ap4e1 KO mouse models recapitulate characteristic neuroanatomical phenotypes of AP-4 deficient patients. This Ap4e1 KO mice have reduced levels of the AP4B1 subunit, indicating destabilization of the entire AP-4 complex. Autophagy-related 9A (ATG9A), an AP-4 cargo protein critical for autophagosome biogenesis, is retained within the trans-Golgi network (TGN). This retention results in depletion of axonal ATG9A and impaired axonal integrity in AP-4 deficiency syndrome. Gene therapy with the AAV9/AP4M1 described herein is expected to ameliorate deficits in Ap4 ml deficient mice.

Ap4 ml KO mouse model was generated through targeted mutation 1b by Wellcome Trust Sanger Institute and recovered by Jackson Laboratories. The phenotypes of this model were not characterized. However, Ap4e1 KO mouse model exhibits a range of neurological phenotypes, including hindlimb clasping, decreased motor coordination, and weak grip strength. Considering the same physiological function of AP4E1 and AP4M1, Ap4 ml KO mice should exhibit similar neurological phenotypes. In this efficacy study, Ap4 ml KO cohorts were be monitored for the presence or absence and the onset of neurological signs following AAV9/AP4M1 gene therapy. The experimental therapy was expected to delay, attenuate, or prevent the onset of symptoms in the treated groups dependent upon the level and time of intervention.

The purpose of this study was to evaluate the efficacy of AAV9/AP4M1 to ameliorate deficits in a mouse model of SPG50.

The experimental design of the in vivo efficacy study with AAV9/AP4M1 in SPG50 (Ap4 m1 KO) mice is provided in Table 1. In brief, homozygous knock-out (KO) Ap4 m1(−/−) male and female mice were administered AAV9/AP4M1 at 1.25E11, 2.5E11, or 5E11 vg/animal, or equivalent volume of vehicle (veh) solution in 5 uL bolus via lumbar intrathecal (IT) injection at post-natal day (p)7-10 or p90. A wild-type (WT) Ap4 ml(+/+) group and a heterozygous (Het) Ap4 m1(+/−) group without treatment were included as controls (Table 1).

TABLE 1
21-month efficacy study in Ap4ml KO mice (ongoing)

| | | Dose | Number of | | |
|---|---|---|---|---|---|
| Ap4ml | AAV9/AP4M1 | Group | Animals | | Time of |
| Allele | Dose | (Name) | (Male/Female) | | Dosing |
| Ap4ml(+/+) | na | A (WT) | 12 | (7/5) | na |
| Ap4ml(+/−) | na | B (Het) | 45 | (23/22) | na |
| Ap4ml(−/−) | Vehicle* | C | 41 | (19/22) | p7-10 or 90 |
| Ap4ml(−/−) | 1.25E11 | D | 16 | (7/9) | p7-10 |
| | vg/animal* | | | | |
| Ap4ml(−/−) | 5E11 vg/animal* | E | 21 | (8/13) | p7-10 |
| Ap4ml(−/−) | 1.25E11 | F | 17 | (10/7) | p90 |
| | vg/animal* | | | | |
| Ap4ml(−/−) | 2.5E11 vg/animal* | G | 22 | (9/13) | p90 |
| Ap4ml(−/−) | 5E11 vg/animal* | H | 19 | (8/11) | p90 |

*Animals dosed via lumbar IT injection. WT, Wild type. Het, Heterozygous. KO, Knockout.

Mice were monitored for changes in body weight, clinical signs, adverse events, and mortality following treatment. All mice were weighed weekly for the first month and then monthly thereafter. Any clinical signs or adverse events including neurological symptoms were investigated, evaluated, and recorded. Appropriate supportive or therapeutic interventions were offered per Institutional Animal Care and Use Committee (IACUC) and veterinary guidance. Three weeks post injection, 6 mice from each group (n=6) were euthanized. Mouse brains were used for AP4M1 mRNA expression by RNAscope and mouse serum was used to check serum toxicity panel including Aspartate transaminase (AST), Total bilirubin (TBIL), Albumin (ALB), Creatine Kinase (CK), and Blood Urea Nitrogen (BUN). Splenocytes from mouse spleen were used in ELISpot assays to detect any immune responses to either AAV9 or transgene. Performance in a battery of behavioral tests were assessed at 3, 5, 8, 12, and 18 months of age compared to homozygous control littermates. Blood and tissue samples were collected from mice that were euthanized for humane reasons. Terminal serum and tissue samples at 21 months old were collected for serum toxicity panel and histopathological assessment, respectively.

The assessments at 8 months indicate that AAV9/AP4M1 dose dependently increased AP4M1 mRNA expression; induced minimal immune responses; caused minimal toxicity 3 weeks post injection; generated minimal effects on body weight; created minimal effects on survival; improved abnormal behaviors.

### Example 2: Material and Methods

### Material:

The vector used was scAAV9/UsP-hAP4M1opt-BGHpA, at a 1.03E¹⁴ vg/ml stock concentration. The quality was assessed and reached research grade quality. PBS containing 5% sorbitol was used. Hamilton Syringe (50 uL, model 705 RN, product #7637-01), Hamilton R N custom needle (length 0.5″, point style #4, 12, 30-gauge, product #7803-07) were used for administration.

Test system was a transgenic mouse model of SPG50 disease. C57BL/6N-Ap4m1tm1b(EUCOMM)Wtsi, recovered by Jackson Laboratories from European Mouse Mutant Archive. There were 22 to 41 animals in KO groups included in this study; 12 WT and 45 Het untreated controls were also included. Total number of animals was 193. Both male and female animals were included in this study.

Animals dosed either between p7-10 (pre-symptomatic), or at p90 (early-symptomatic). Intrathecal (IT) injection of AAV9/AP4M1 to mice at p7-10 pre-symptomatic stage might prevent and/or delay the onset of symptoms, whereas IT injection of AAV9/AP4M1 to mice at p90 early-symptomatic stage might slow, stop, or reverse the progression. These studies support the justification for a potential human trial by following the dose timing (i.e., after diagnosis) and rout of administration that will be used in clinical trial patients.

The mouse model recapitulates the complex in vivo environment that AAV9/AP4M1 will encounter, enabling an assessment of rescue and potential side effects in multiple areas of the nervous system relevant for treatment of the disease.

Animals were administered treatment AAV9/AP4M1 or vehicle via acute intrathecal (IT) lumbar injection. AAV9/AP4M1 were injected IT in each animal by a qualified lab member, in a volume of 5 uL as a bolus injection.

Mice were randomized into treatment groups based on the ID's assigned to them at genotyping (Table 1).

### Experimental Protocol:

Besides regular monitoring of body weight, clinical signs, adverse events, and mortality following the treatment, all mice underwent a behavioral testing battery, including Hindlimb Clasping, Rotarod, Grip Strength, Wire Hang, Open Field, and Elevated Plus Maze. Tests were selected based on suspected deficits in the Ap4 ml KO mouse model, as well as for their ability to be repeated for longitudinal testing. These behavioral tests will be/were repeated at 3, 5, 8, 12 and 18 months of age. All behavior tests were conducted by personnel blind to the genotype and treatment of the mice.

Three weeks post injection, 6 mice from each group were euthanized. Mouse brains were used for AP4M1 mRNA expression by RNAscope and mouse serum was used to check serum toxicity panel including AST, TBIL, ALB, CK, and BUN. Splenocytes from mouse spleen were used in ELISpot assays to detect any immune responses to either AAV9 or transgene. Terminal serum and tissue samples at 21 months old will be collected for serum toxicity panel and histopathological assessment, respectively.

### Plasmid Design and Development:

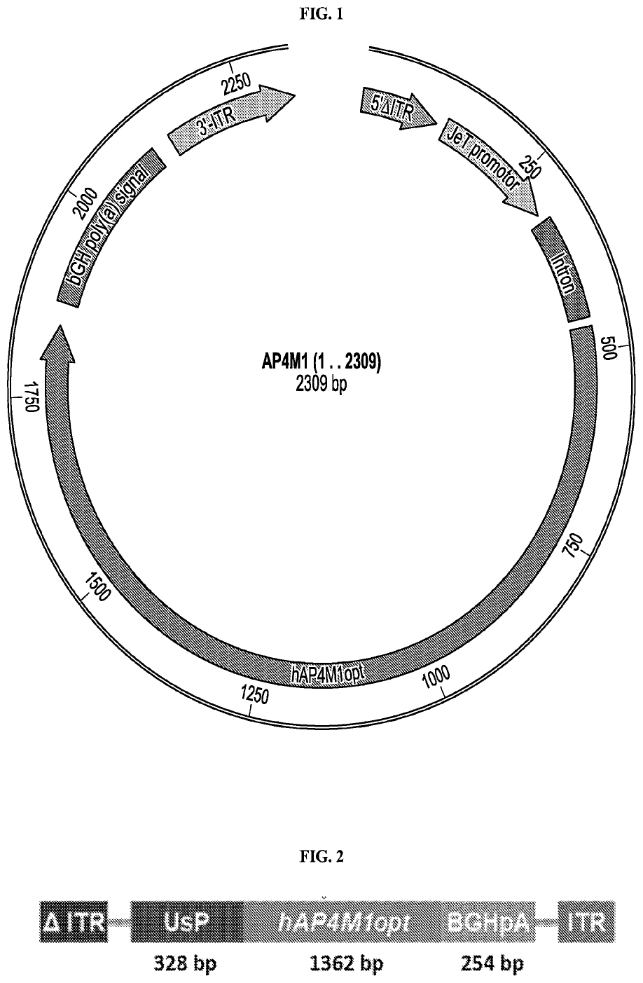

AAV9/AP4M1 was designed and developed with a plasmid containing the transgene of a human AP4M1 codon-optimized construct (hAP4M1opt, see FIG. 1). The DNA sequence was modified such that the final amino acid sequence is unchanged, but the transgene sequence is distinct from the endogenous gene to enable specific transgene detection by various molecular methods. The transgene consists of a human AP4M1 DNA coding sequence between a promoter and a polyadenylation signal. Sanger sequencing was used to confirm the plasmid sequence, as seen in FIG. 2; on the schematic diagram the AP4M1 construct cassette comprises a mutant AAV2 inverted terminal repeat (ITR) with the D element deleted (A ITR), the UsP promoter, the human AP4M1 codon-optimized coding sequence (hAP4M1opt), the BGHpA signal, and WT AAV2 ITR.

### AAV9/AP4M1 Vector Preparation:

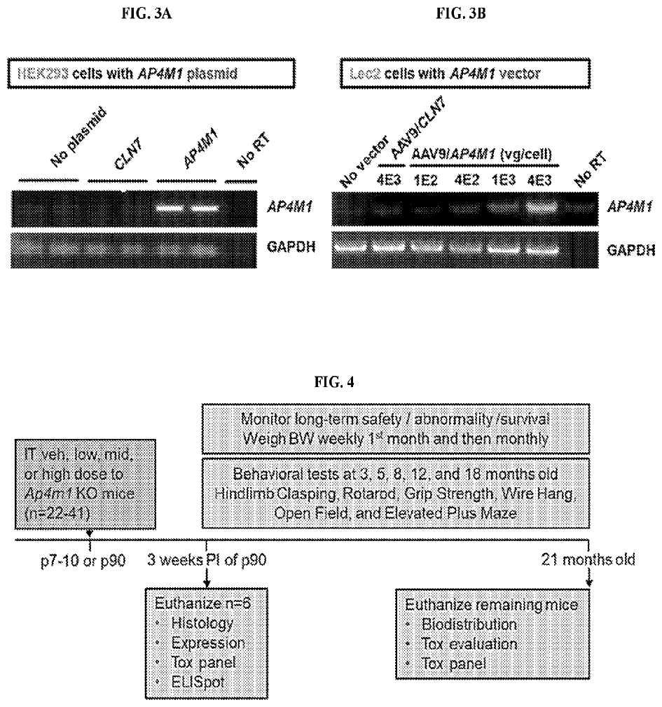

The established plasmid was packaged into self-complementary (sc)AAV9 vectors which are 10-100 times more efficient at transduction compared to traditional single-stranded (ss)AAV vectors. scAAV9 vectors were produced by UNC-VC. The final research-grade products were dialyzed in phosphate-buffered saline (PBS) with additional 212 mM NaCl and 5% D-sorbitol, tittered by qPCR, and confirmed by silver stain. RT-PCR was used to confirm AP4M1 mRNA expression in HEK293 cells following transduction with AP4M1 plasmid (FIG. 3A) and in Lec2 cells following transduction with AAV9/AP4M1 (FIG. 3B).

As seen in FIG. 3A, AP4M1 mRNA expression were measured by RT-PCR in HEK293 cells transduced without plasmid, with CLN7 (disease-irrelevant transgenes), or AP4M1 (therapeutic transgene) plasmid. In FIG. 3B, AP4M1 mRNA expression were measured by RT-PCR in Lec2 cells transduced without vector, with CLN7 (disease-irrelevant transgenes), or AP4M1 (therapeutic transgene at increasing doses) vector. GAPDH was used as an internal control.

### Ap4 m1 Ko Mice:

Ap4 m1 KO mouse model was generated through targeted mutation 1b by Wellcome Trust Sanger Institute and recovered by Jackson Laboratories. The Ap4 m1 KO mice are identified by toe tattooing at p7-10 and then randomized into treatment groups based on the ID numbers assigned to them at genotyping. Genotyping was performed using genomic DNA extracted from clipped tail and four primers:

(1) FGEN-AP4M1 WT
SEQ ID NO: 8
(TAGAAGCCAGCCATAGTGGT),
(2) RGEN-AP4M1 WT
SEQ ID NO: 9
(GAGCAAGTGTGTGGACCTGA),
(3) FGEN_AP4M1 mut
SEQ ID NO: 10
(CGGTCGCTACCATT ACCAGT),
and
(4) RGEN-AP4M1 mut
SEQ ID NO: 11
(TGTTTCAGTGTCCCATCTGC).

A PCR protocol with annealing temperature of 65° C. for 30 cycles is used for genotyping, which generates wild type band of 355 bp and mutant band of 435 bp, respectively.

### Efficacy Study Plan in Ap4 m1 KO Mice:

The mouse study plan for in vivo efficacy is summarized in FIG. 4. In brief, both male and female Ap4 m1 KO mice were randomized into treatment cohorts and injected IT at p7-10 (pre-symptomatic cohorts) or p90 (early-symptomatic cohorts). For IT, 5 μL of vehicle or 1.25E11, 2.5E11, or 5E11 vg/mouse of AAV9/AP4M1vector was administrated via lumber puncture. All mice were weighed weekly up to 4 weeks old and monthly thereafter, as well as observed for overt signs of adverse effects at the times of weighing. The survival rate was calculated, and all unexpected deaths were investigated by a veterinary staff. Starting at 3 months of age, behavioral testing was carried out on all study cohorts and then repeated at 5-, 8-, 12-, and 18-months post dosing. A battery of behaviors, focusing mainly on motor function and including Hindlimb Clasping, Rotarod, Grip Strength, Wire Hang, Open Field, and Elevated Plus Maze, was assessed blindly through the mouse behavior phenotyping core facility. At 3 weeks post injection, 3 males and 3 females from each cohort were sacrificed to evaluate hAP4M1opt mRNA expression by RNAscope and early histological signs of treatment efficacy. Mouse serum was used to check serum toxicity panel including AST, TBIL, ALB, CK, and BUN. Splenocytes from mouse spleen were used in ELISpot assays to detect any immune responses to either AAV9 or transgene. All remaining mice will be maintained to evaluate long-term survival and safety until 21 months old, when necropsy, histopathology, and toxicity evaluation are performed.

### Standard Operating Procedure for Intrathecal AAV Delivery in Awake Mice:

Materials: 50 ul Hamilton syringe (Hamilton, 7637-01); 30-gauge Hamilton needle (Hamilton, 7803-07); Gauze; Micropipette; Pipet tips; AAV to be injected; Hamilton cleaning solution (Hamilton, HT 18311); 70% ethanol; ddH2O.

### Methods:

1. Prepare the materials listed above.
2. Wipe the working area in a hood with NPD provided by ARC.
3. Assemble the 50 ul Hamilton syringe with a 30-gauge Hamilton needle.
4. Draw 5 ul of AAV solution into the syringe gently, but make sure to remove air bubbles.
5. Put the awake mouse in a prone position in the hood.
6. Cover the upper body with sterile gauze to calm the mouse and also to avoid being bitten.
7. Fix the mouse by gripping firmly on its pelvic girdle with the thumb and the index fingers and hold gently on the upper body of the mouse with the palm.
8. Feel the intervertebral space along the middle line and press an indentation with a fingernail to indicate the L5-L6 intervertebral space.
9. Rotate the base of the tail slightly and gently to indicate the midline of the spine.
10. Insert the needle vertically in the intersection of the indentation.
11. Reduce the angle to approximately 300 slowly when the needle attaches the bone.
12. Slip the needle into the intervertebral space.
   a. Sudden tail flick is a sign of successful entry into the intradural space.
   b. Lack of lateral mobility of the needle is an indication it is in intradural space.
13. Inject the vector solution within 15 seconds and retain the needle approximately 15 seconds.
14. Withdraw the needle with gentle rotation to avoid leaking.
15. Monitor the mouse post-injection for a return to normal ambulation.
   a. Monitor the mouse for impairment in breathing, poor body condition, posture, coordination, mobility or any motor function impairment.
   b. These clinical signs if noticed in the first 24 hours of lumbar puncture are attributable to a spinal injury and the mice will be taken off the study and euthanized.
16. Return the mouse back to the cage.
17. Clean the bench with NPD
18. Clean the syringe 10× Hamilton cleaning solution, 10×70% ethanol, and 10× ddH2O.
19. Dissemble and rinse the syringe with water and wipe the parts with clean tissue paper.
20. Save the needle and syringe.

### Tissue Processing and Sectioning and RNAscope:

At necropsy, animals were deeply anesthetized via an intraperitoneal injection of 2.5% avertin solution in normal saline. Animals were perfused for 5 minutes with 1×PBS containing 1 unit/mL heparin. Brain and spinal cord were harvested and fixed in 10% neutral-buffered formalin (NBF) for 24 hours and transferred to 70% ethanol. Tissues were then processed, embedded in paraffin, and cut into 5 μm sections on slides.

For RNAscope to evaluate human AP4M1 mRNA expression, slides were deparaffinated with xylene, which was removed with 200 proof ethanol, incubated with Hydrogen Peroxide for 10 minutes at room temperature, and then washed with distilled water. The slides were boiled in 1× Target Retrieval solution for 10 minutes, washed with distilled water, dehydrated with 200 proof ethanol, and allowed to air dry. Following this antigen retrieval, Protease Plus was added to each section and incubated at 40° C. for 30 minutes. The slides were washed with distilled water, incubated with Hs-Codon-CLN7 RNAscope probe in HybEZ Oven for 2 hours at 40° C., and washed with 1× Wash Buffer. The slides were incubated with AMP 1-6 for 15 minutes following RNAscope 2.5 HD Detection Kit protocol and then with RED solution for 5 minutes followed by counter stained with Mayer's hematoxylin.

### Image Analysis:

All stained slides were digitized with a ScanScope slide scanner (Aperio Technologies). Scanned slides were viewed with the ImageScope software package (Version 10.0, Aperio Technologies) and analyzed using custom analysis settings in HALO™ Image Analysis Platform (Halo2.2, Indica Labs). A region of interest (ROI) was hand drawn on each image to allow for analysis by tissue region. Within the brain, regions were drawn around the cortex, sub-cortex, cerebellum, and brain stem. For spinal cord samples, the entire tissue area was analyzed. Threshold for each stain was set using positive and negative control images, and the same analysis settings was applied for every image of the same stain. Percent area strongly staining for each marker of interest was recorded for each tissue/ROI. Analysis was done with the observer blinded to treatment group of each sample.

### Splenocyte Preparation:

During necropsy, approximately half of the spleen from each animal was placed in a well of a 24-well plate containing 1 mL pre-chilled complete RPMI-1640 (cRPMI-1640) medium. The spleen was transferred onto a 70 μm cell strainer sitting on a 50 mL conical tube and crushed/torn apart using the rubber-plunger of a sterile 1 mL syringe. The splenocytes were collected by running 10 mL of cRPMI-1640 medium through the strainer and centrifuged at 400×g for 10 minutes at 4° C. The splenocytes were then resuspended in 1 mL of ACK lysis buffer (Gibco A1049201) to lyse red blood cells. After 10 minutes of lysis the reaction was stopped by the addition of 9 mL of chilled cRPMI-1640 medium and centrifuged again. Splenocytes were then resuspended, counted, placed in freezing medium (90% FBS, 10% DMSO) at 2E7 cells/mL, frozen, and placed in the vapor phase of liquid nitrogen until they were analyzed via ELISpot.

### Preparation of Peptide Library Pools for AAV9 Capsid or AP4M1 Protein:

Both peptide library pools of AAV9 capsid and AP4M1 protein were purchased from Mimotopes, Victoria, Australia. Both libraries were comprised of 15-mers with a 5 amino acid offset. The AAV9 capsid library pool contained 145 peptides and the final concentration of each peptide in the pool was 0.63 mg/mL in 1.59% DMSO final. The AP4M1 peptide library pool contained 89 peptides and the final concentration of each peptide in the pool was 1.02 mg/mL in 0.61% DMSO final. Both pools were stored at −80° C. before use.

ELISpot assays:

ELISpot assays were performed using an ImmunoSpot kit (mIFNg-1M/5, Cellular Technology Limited). Briefly, splenocytes were thawed, washed, and resuspended in cRMPI-1640 medium for counting. 2E5 splenocytes in 100 μL of cRPMI-1640 medium were plated into each well of an ELISpot plate in quintuplicates whenever possible. 100 μL of cRPMI-1640 medium containing AAV9 capsid pool or AP4M1 peptide pool was then added to the wells. The controls included cells with no peptide, cells stimulated with a mixture of Phorbol 12-myristate 13-acetate (PMA) and Ionomycin, (Invitrogen, 00-4970-93), medium with INFγ which was supplied with the kit, or medium only. The splenocytes were incubated for 48 hours in a humidified 37° C. CO2 incubator. All other steps were performed according to the manufacturer's recommendation.

### Behavior Tests:

For Hindlimb Clasping testing, mice were suspended by the tail for 10 seconds at 20 cm height from the procedure table and the posture of hindlimbs was visually examined and recorded. The response was considered clasping if one (score 1) or both (score 2) hindlimbs retracted and touched the abdomen for more than 5 seconds during the suspension. If the hindlimbs remain splayed outward during the entire time of suspension, the mouse was designated as non-clasping (score 0).

For Rotarod testing, mice were placed on a stationary rotarod (Columbus Instruments). The rod was then accelerated from 4 to 40 rpm over 5 min. The time that each mouse falls from the rod was recorded. If a mouse holds onto the rod and rotates completely around, he was treated as if he has fallen from the rod at that time. Each mouse was tested four times a day for two consecutive days with at least a 15 min intertrial interval.

For Grip Strength testing, mice were weighed, and tail marked. All males tested first, then females to minimize issues for the males. All animals in a cage tested before moving to next cage. Apparatus (San Diego Instruments) was cleaned with 70% ethanol between mice. Mice were individually placed onto the resting platform and allowed to explore for ˜30 seconds. Trials were conducted by lifting the mouse by the base of tail, allowing it to grasp the grid at a 45-degree angle, and pulled in a horizontal plane until they released the grid. Peak force value was recorded. Inter-trial interval was a minimum of 15 seconds. Trials in which the mouse was directly seen to have improper/inadequate grasp during the pull phase were excluded. Testing was stopped if paw injuries are detected.

For Wire Hang testing, mice were placed on a metal screen and the screen were shaken gently so the mice grab on. The screen was then inverted approximately 30 cm over a mouse cage (with bedding about 2-3 inches thick). The time until they released from the wire were recorded. The test was repeated 3 times with approximately 30 min between each trial.

For Open Field testing, mice were placed in the periphery of a novel open field environment (44 cm×44 cm, walls 30 cm high) in a dimly lit room (approximately 60 lux) and allowed to explore for 10 min. The animals were monitored from above by a video camera connected to a computer running video tracking software (Ethovision XT, version13.0, Noldus, Leesburg, Virginia) to determine the time, distance moved and number of entries into three areas: the periphery (5 cm from the walls), the center (14 cm×14 cm) and the non-periphery (the whole arena excluding the periphery zone). The open field arenas were wiped and allowed to dry between mice.

For Elevated Plus Maze testing, mice were placed in the center zone of an elevated maze raised 100 cm from the ground. The maze contained two open arms and two closed arms of equal width and length. All mice were placed in the same central point of the maze and allowed to freely explore for 5 min. Animal activity was monitored using video tracking software (Ethovison 13.1, Noldus). Duration in the open arms, as well as total distance moved, were tracked and recorded. The maze was wiped thoroughly with disinfectant between animals.

### Animal Management:

Animals were monitored after dosing for impairment in breathing, poor body condition, posture, coordination, mobility, or any motor function impairment. If these clinical signs were noticed in the first 24 hours of lumbar puncture, they were attributed to a spinal injury and the mice were taken off the study and euthanized. No animal was excluded from the study due to spinal injury. Animals were weighed and observed for adverse health effects at 1-, 2-, 3-, and 4-weeks post-injection, and monthly thereafter.

### Statistical Evaluation:

All quantitative data were presented as mean±SEM, analyzed, and graphed using GraphPad Prism Software (v. 9.2.0, GraphPad Software). A ROUT test was used first to remove any outlier. Data were then tested for normal distribution (Shapiro-Wilk normality test) and homogeneity of variance (Brown-Forsythe test). Data sets that passed these two tests were analyzed using Student's unpaired t-test for two group comparison or one-way ANOVA for equal or more than three groups comparison with Dunnett's correction for relevant pairwise comparisons to KO-Veh group. Data sets that did not pass tests for normality or homogeneity of variance were analyzed using Mann-Whitley test for two group comparison or Kruskal-Wallis test with Dunn's correction for relevant pairwise comparisons to KO-Veh group. For survival analysis, data showing in Kaplan-Meier survival curve were compared with Log-rank (Mantel-Cox) test. Two-way ANOVA with repeated measures was used for body weight analyses. A p<0.05 was considered as significant for all statistical analyses.

### Example 3: Results and Discussion

AAV9/AP4M1 Increased hAP4M1Opt mRNA Expression in Brain Regions 3 Weeks Post Injection in Ap4 m1 KO Mice

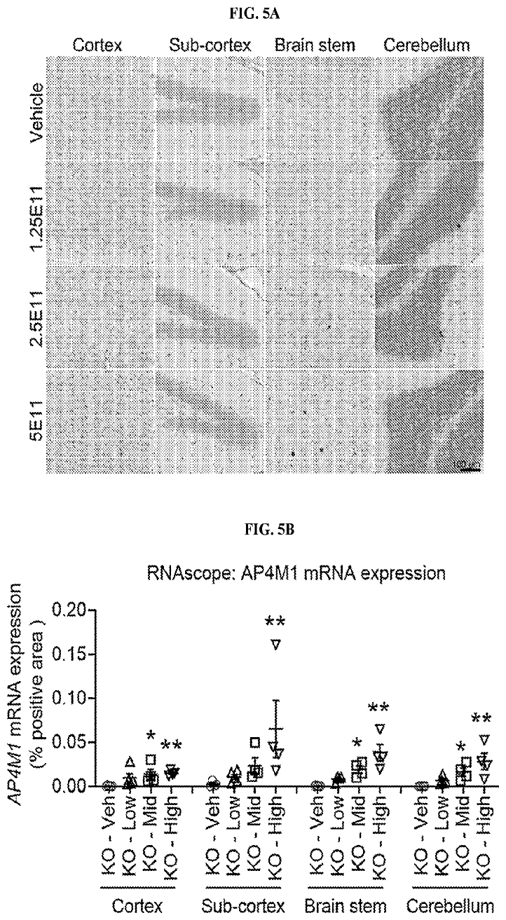

Animals receiving AAV9/AP4M1 had detectable levels of hAP4M1opt mRNA in all brain regions assessed (FIGS. 5A and 5B). The high dose group had significantly higher mRNA levels than KO-Veh control animals. Positive hAP4M1opt mRNA staining by RNAscope is observable as a deep red color.

Percent area staining positive for hAP4M1opt mRNA by brain regions. Each data point represents measurement from an individual animal, with lines representing the mean measurement±SEM (n=5−8/group). **p<0.01 compared to KO-Veh.

### AAV9/AP4M1 Induced Minimal Immune Responses in Ap4 ml KO Mice

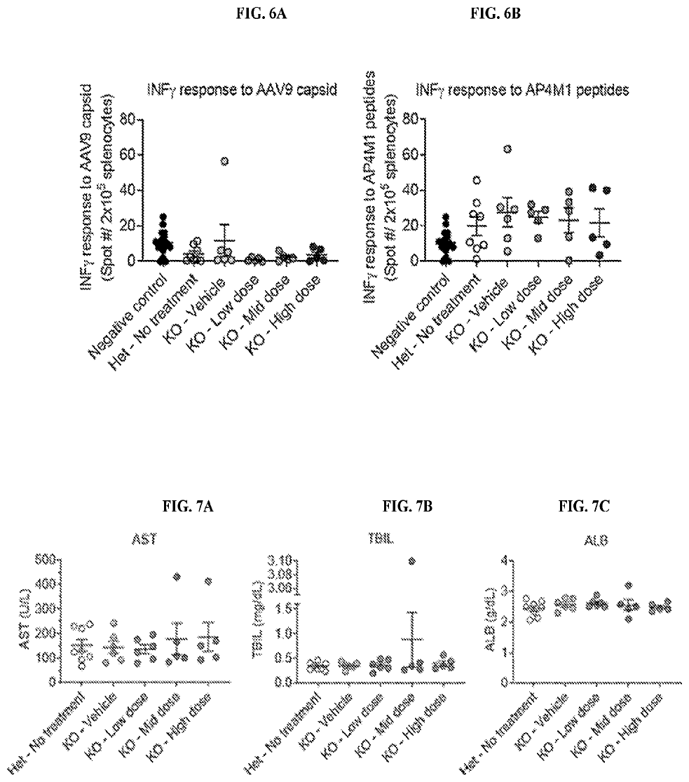

Immune responses to AAV and/or transgene remain a major challenge in translating experimental drugs to approved medicine. While natural AAV infection can prevent patients from receiving AAV gene therapy, immune responses to AAV gene therapy can result in loss of transgene expression and even tissue damage. To evaluate the INFγ immune response to AAV9/AP4M1 in the Ap4 m1 KO mouse model, splenocytes from mice treated with AAV9/AP4M1 3 weeks prior were plated and treated in vitro with either AAV9 capsid or AP4M1 peptide pools for 2 days along with both negative (no peptide) and positive (PMA+Ionomycin) controls. The controls behaved normally (i.e., the negative control had roughly 10 spots and the positive control had too many spots to count), and the splenocytes from treated mice did not show a spotting pattern suggestive of a T-cell response against AAV9 or AP4M1. Together, these results suggest that the AAV9/AP4M1 induced minimal INFγ immune response to either AAV9 or the human AP4M1 protein in the Ap4 ml KO mouse model. Specifically, the results support the notion that the codon-optimization of AP4M1 does not create any new immunogenicity for the expression AP4M1 protein. As shown in FIGS. 6A and 6B, where spot numbers in each well were blindly counted with a specialized automated ELISpot reader, each data point represents the mean value of duplicate assay for negative control or the mean value of quintriple assay from an individual animal, with lines representing the mean measurement±SEM (n=5-8/group). No significance was found compared to KO-Veh group.

Spot numbers in each well were blindly counted with a specialized automated ELISpot reader. Each data point represents the mean value of duplicate assay for negative control or the mean value of quadruplicate assay from an individual animal, with lines representing the mean measurement±SEM (n=5-8/group). No significance was found compared to KO-Veh group.

### AAV9/AP4M1 Caused Minimal Toxicity 3 Weeks Post Injection in Ap4 ml KO Mice

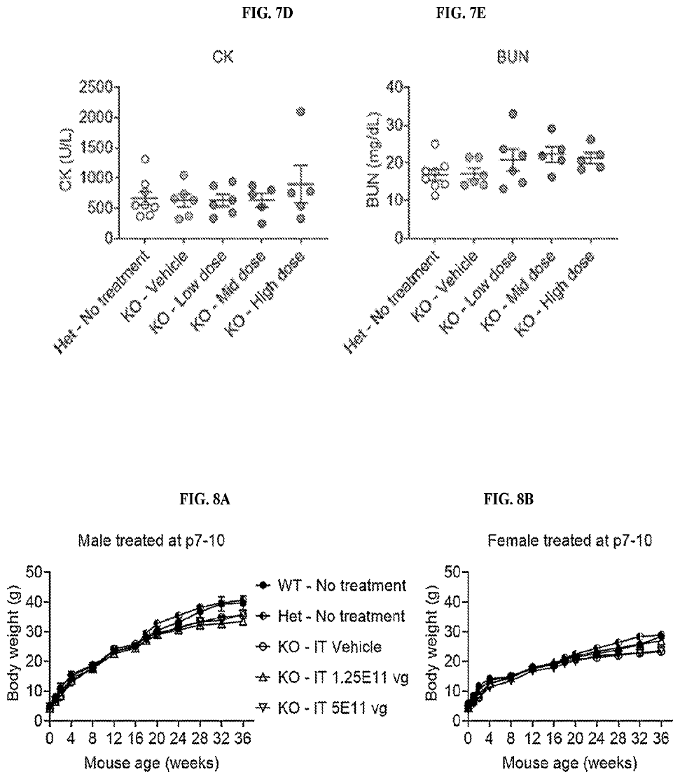

During necropsy, mouse serum was collected for the measurement of serum toxicity panel including AST (FIG. 7A), TBIL (FIG. 7B), ALB (FIG. 7C), CK (FIG. 7D), and BUN (FIG. 7E). Animals receiving AAV9/AP4M1 had normal levels of serum toxicity panel 3-weeks post injection in all except 1 mouse in mid dose and 1 mouse in high dose group (FIGS. 7A-7E). The mouse received mid dose of 2.5E11 vg/mouse had serum AST and TBIL levels of 431 U/L and 3.1 mg/dL, respectively. The mouse received high dose of 5E¹¹ vg/mouse had serum AST and CK levels of 413 U/L and 2102 U/L, respectively. In conclusion, our results suggest that most of the Ap4 m1 KO mice tolerate AAV9/AP4M1 well, however there is an occasional toxicity to the liver and muscles.

Either high (5E11 vg/mouse), mid (2.5E11 vg/mouse), or low (1.25E11 vg/mouse) dose of AAV9/AP4M1 was administered IT to Ap4 m1 KO mice at p90. At 3-weeks post injection, mouse serum was collected for serum chemistry. Each data point represents measurement from an individual animal, with lines representing the mean measurement±SEM (n=5-8/group). No significant difference was found compared to KO-Veh group.

### AAV9/AP4M1 Generated Minimal Effects on Body Weight

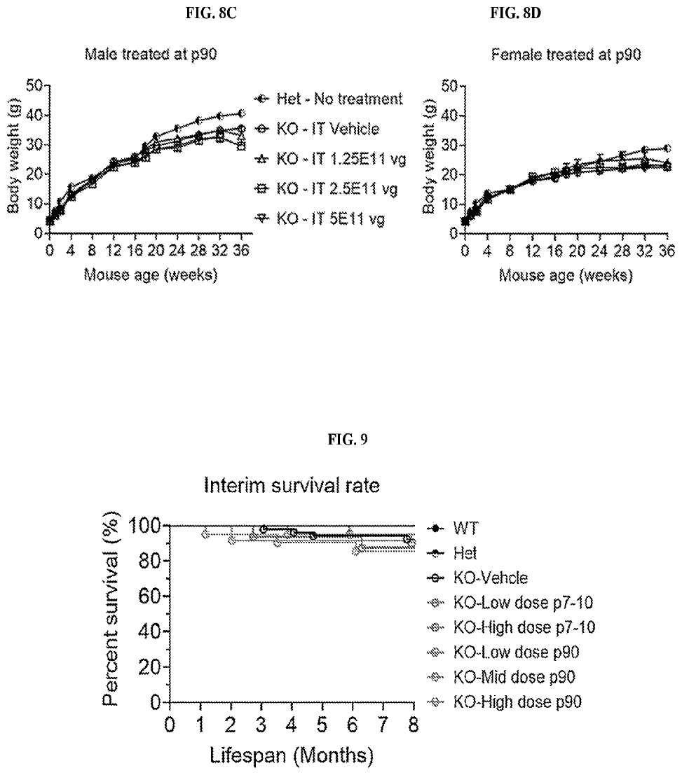

Either high (5E11 vg/mouse), mid (2.5E11 vg/mouse), or low (1.25E11 vg/mouse) dose of AAV9/AP4M1 was administered IT to Ap4 ml KO mice at p7-10 or p90. Body weight was taken longitudinally. Results are presented as mean±SEM (n=12-45/group). No significant difference was found up to 8 months old compared to KO-Veh group (see FIGS. 8A, 8B, 8C and 8D).

### AAV9/AP4M1 Created Minimal Effects on Survival

Kaplan-Meier survival curve shows the survival over 8 months lifespan. Data were compared with Log-rank (Mantel-Cox) test. No significant difference was found compared to KO-Veh group (see FIG. 9).

### AAV9/AP4M1 Improved Abnormal Behaviors

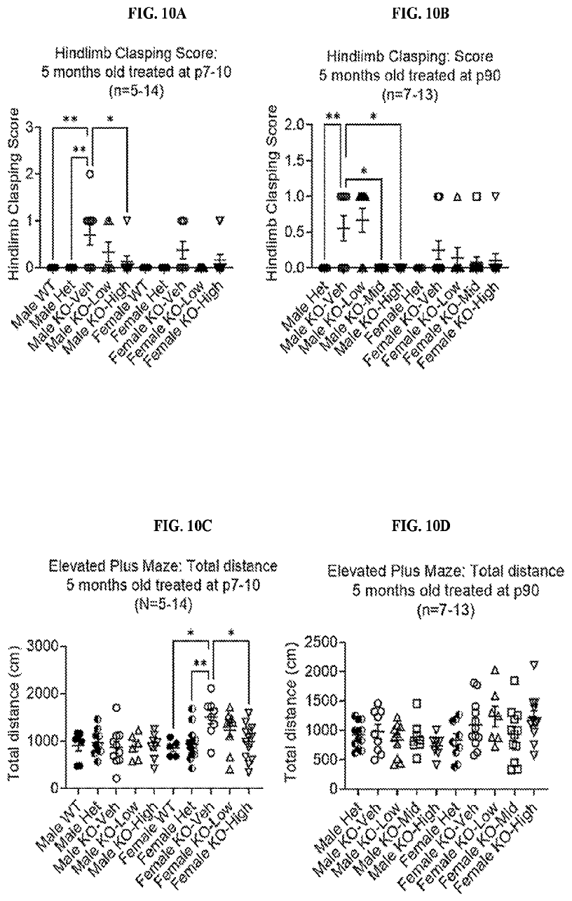

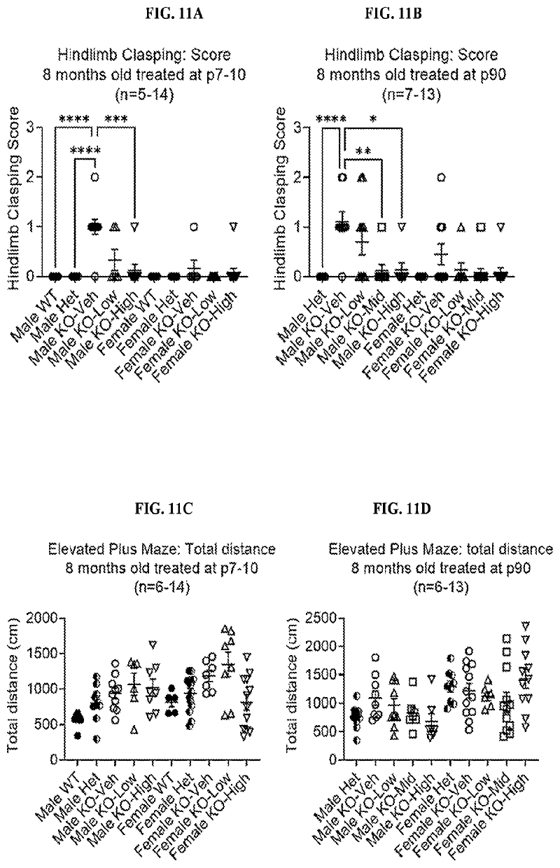

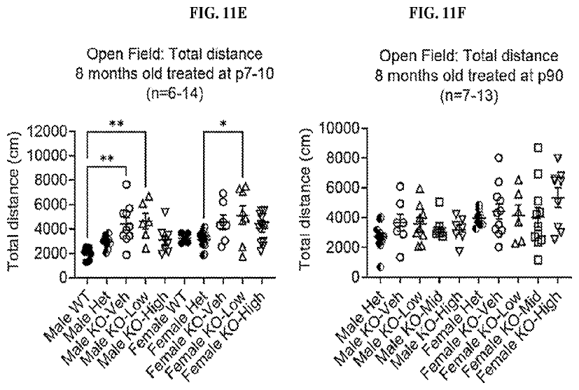

No behavioral deficits were observed between WT or Het control animals and KO-Veh or KO-AAV9/AP4M1 dosed animals at 3 months. At 5 months old, KO-Veh male but not female animals started to show significantly increased Hindlimb Clasping scores compared to WT or Het control animals, which was improved by treating the animals with high dose AAV9/AP4M1 at p7-10, or mid and high dose AAV9/AP4M1 at p90 (FIGS. 10A and 10B). For Elevated Plus Maze testing, KO-Veh female but not male animals started to show significantly increased total distance travelled compared to WT or Het control animals, which was improved by treating the animals with high dose AAV9/AP4M1 at p7-10, but not by mid and high dose AAV9/AP4M1 at p90 (FIGS. 10C and 10D). Similar patterns were seen at 8 months old except more severe Hindlimb Clasping score (FIGS. 11A and 11B) and less severe total distance on both Elevated Plus Maze (FIGS. 11C and 11D) and Open Field in KO-Veh animals (FIGS. 11E and 11F). Rotarod, Grip Strength, and Wire Hang tests did not detect any phenotypes in KO-Veh compared to WT or Het control animals up to 8 months old.

Hindlimb Clasping (FIGS. 10A and 10B) and Elevated Plus Maze performance (FIGS. 10C and 10D) of mice treated at p7-10 (left panels) and p90 (right panels). Each data point represents measurement from an individual animal, with lines representing the mean measurement±SEM (n=5-14/group). *p<0.05 and **p<0.001 compared to KO-Veh.

Hindlimb Clasping (FIGS. 11A and 11B), Elevated Plus Maze (FIGS. 11C and 11D), and Open Field (FIGS. 11E and 11F) performance of mice treated at p7-10 (left panels) and p90 (right panels). Each data point represents measurement from an individual animal, with lines representing the mean measurement±SEM (n=5-14/group). *p<0.05, **p<0.001, ***p<0.001, and ****p<0.0001 compared to KO-Veh.

### Example 4: Conclusions

Interim results for the initial 8 months demonstrated that AAV9/AP4M1 dose dependently increases AP4M1 mRNA expression (FIG. 5A), induces minimal immune responses (FIGS. 6A-6B), causes minimal toxicity (FIGS. 7A-7E), generates minimal effects on body weight (FIGS. 8A-8D), creates minimal effects on survival (FIG. 9), and improves abnormal behaviors (FIGS. 10A-10D and FIGS. 11A-11F). The high dose of 5E11 vg provided maximal pharmacological effects on Hindlimb Clasping whether administered early on p7-10 or p90 in male mice. For the Maze total distance, female mice had a less robust response when dosed at p90 compared to p7-10. The mid dose of 2.5E11 vg provided some pharmacological effects on Hindlimb Clasping when administered p90 in male mice, suggesting AAV9/AP4M1 may also generate benefits when dosed at the early-symptomatic stage.

### REFERENCES

1. Robinson M S. Adaptable adaptors for coated vesicles. Trends in cell biology. 2004; 14(4):167-74.
2. Hirst J, Irving C, and Borner G H. Adaptor protein complexes AP-4 and AP-5: new players in endosomal trafficking and progressive spastic paraplegia. Traffic (Copenhagen, Denmark). 2013; 14(2):153-64.
3. Abou Jamra R, Philippe O, Raas-Rothschild A, Eck S H, Graf E, Buchert R, et al. Adaptor protein complex 4 deficiency causes severe autosomal-recessive intellectual disability, progressive spastic paraplegia, shy character, and short stature. American journal of human genetics. 2011; 88(6):788-95.
4. Verkerk A J, Schot R, Dumee B, Schellekens K, Swagemakers S, Bertoli-Avella A M, et al. Mutation in the AP4M1 gene provides a model for neuroaxonal injury in cerebral palsy. Americanjournal of human genetics. 2009; 85(1):40-52.
5. Ivankovic D, Drew J, Lesept F, White I J, Lopez Domenech G, Tooze S A, et al. Axonal autophagosome maturation defect through failure of ATG9A sorting underpins pathology in AP-4 deficiency syndrome. Autophagy. 2019:1-17.
6. De Pace R, Skirzewski M, Damme M, Mattera R, Mercurio J, Foster A M, et al. Altered distribution of ATG9A and accumulation of axonal aggregates in neurons from a mouse model of AP-4 deficiency syndrome. PloS genetics. 2018; 14(4): e1007363.
7. Davies A K, Itzhak D N, Edgar J R, Archuleta T L, Hirst J, Jackson L P, et al. AP-4 vesicles contribute to spatial control of autophagy via RUSC-dependent peripheral delivery of ATG9A. Nature communications. 2018; 9(1):3958.
8. Hardies K, May P, Djemie T, Tarta-Arsene O, Deconinck T, Craiu D, et al. Recessive loss-of-function mutations in AP4S1 cause mild fever-sensitive seizures, developmental delay and spastic paraplegia through loss of AP-4 complex assembly. Human molecular genetics. 2015; 24(8):2218-27.
9. Gray S J, Foti S B, Schwartz J W, Bachaboina L, Taylor-Blake B, Coleman J, et al. Optimizing promoters for recombinant adeno-associated virus-mediated gene expression in the peripheral and central nervous system using self-complementary vectors. Human gene therapy. 2011; 22(9):1143-53.
10. McCarty D M, Monahan P E, and Samulski R J. Self-complementary recombinant adeno-associated virus (scAAV) vectors promote efficient transduction independently of DNA synthesis. Gene therapy. 2001; 8(16):1248-54.
11. McCarty D M, Fu H, Monahan P E, Toulson C E, Naik P, and Samulski R J. Adeno-associated virus terminal repeat (TR) mutant generates self-complementary vectors to overcome the rate-limiting step to transduction in vivo. Gene therapy. 2003; 10(26):2112-8.
12. Gray S J, Choi V W, Asokan A, Haberman R A, McCown T J, and Samulski R J.

Production of recombinant adeno-associated viral vectors and use in in vitro and in vivo administration. Current protocols in neuroscience/editorial board, Jacqueline N Crawley [et al]. 2011; Chapter 4: Unit 4.17.
13. Lykken E A, Shyng C, Edwards R J, Rozenberg A, and Gray S J. Recent progress and considerations for AAV gene therapies targeting the central nervous system. Journal of neurodevelopmental disorders. 2018; 10(1):16.
14. Rabinowitz J, Chan Y K, and Samulski R J. Adeno-associated Virus (AAV) versus Immune Response. Viruses. 2019; 11(2).

What is claimed is:

1. A polynucleotide encoding a human AP4M1 polypeptide, wherein the polynucleotide is codon-optimized for expression in a human cell.

2. The polynucleotide of claim 1, wherein the polynucleotide comprises SEQ ID NO:5, SEQ ID NO:40, or a nucleic acid sequence having at least about 90% identity thereto.

3. An expression cassette comprising the polynucleotide of any one of claims 1-2.

4. The expression cassette of claim 3, wherein the polynucleotide is operably linked to a promoter.

5. The expression cassette of claim 4, wherein the promoter is a JeT promoter or an UsP promoter.

6. The expression cassette of any one of claims 3-5, wherein the polynucleotide is operably linked to an intron.

7. The expression cassette of any one of claims 3-6, wherein the polynucleotide is operably linked to a polyadenylation signal.

8. The expression cassette of claim 7, wherein the polyadenylation signal is a bovine growth hormone polyadenylation signal

9. The expression cassette of any one of claims 3-8, further comprising at least one adeno-associated virus (AAV) inverted terminal repeat (ITR).

10. The expression cassette of claim 9, wherein the expression cassette comprises two AAV ITRs.

11. The expression cassette of claim 10, wherein the two AAV ITRs are identical.

12. The expression cassette of claim 10, wherein the two AAV ITRs are not identical.

13. The expression cassette of any one of claims 3-12, wherein the expression cassette is a self-complementary AAV genome.

14. The expression cassette of any one of claims 3-13, wherein the expression cassette comprises a promoter, a human AP4M1 polynucleotide, and a polyadenylation site.

15. The expression cassette of any one of claims 3-13, wherein the expression cassette comprises an AAV ITR, a promoter, a human AP4M1 polynucleotide, a polyadenylation site, and an AAV ITR.

16. The expression cassette of any one of claims 3-13, wherein the expression cassette comprises a promoter, an intron, a human AP4M1 polynucleotide, and a polyadenylation site.

17. The expression cassette of any one of claims 3-13, wherein the expression cassette comprises an AAV ITR, a promoter, an intron, a human AP4M1 polynucleotide, a polyadenylation site, and an AAV ITR.

18. The expression cassette of claim 14, wherein the expression cassette comprises SEQ ID NO:3, SEQ ID NO:5, and SEQ ID NO:6.

19. The expression cassette of claim 14, wherein the expression cassette comprises SEQ ID NO:40, SEQ ID NO:40, and SEQ ID NO:6.

20. The expression cassette of claim 15, wherein the expression cassette comprises SEQ ID NO:2, SEQ ID NO:3, SEQ ID NO:5, SEQ ID NO:6 and SEQ ID NO:7.

21. The expression cassette of claim 15, wherein the expression cassette comprises SEQ ID NO:2, SEQ ID NO:39, SEQ ID NO:40, SEQ ID NO:6 and SEQ ID NO:7.

22. The expression cassette of claim 16, wherein the expression cassette comprises SEQ ID NO:3, SEQ ID NO:4, SEQ ID NO:5, and SEQ ID NO:6.

23. The expression cassette of claim 16, wherein the expression cassette comprises SEQ ID NO:39, SEQ ID NO:40, and SEQ ID NO:6.

24. The expression cassette of claim 17, wherein the expression cassette comprises SEQ ID NO:2, SEQ ID NO:3, SEQ ID NO:4, SEQ ID NO:5, SEQ ID NO:6, and SEQ ID NO:7.

25. The expression cassette of claim 17, wherein the expression cassette comprises SEQ ID NO:2, SEQ ID NO:39, SEQ ID NO:40, SEQ ID NO:6, and SEQ ID NO:7.

26. The expression cassette of any one of claims 3-25, comprising SEQ ID NO:1, SEQ ID NO: 42 or a sequence at least about 90% identical thereto.

27. A vector comprising the polynucleotide of claim 1 or 2 or the expression cassette of any one of claims 3-26.

28. The vector of claim 27, wherein the vector is a viral vector.

29. The vector of claim 28, wherein the vector is an AAV vector.

30. The vector of claim 29, wherein the AAV vector comprises wild-type capsid proteins.

31. The vector of claim 29, wherein the AAV vector comprises a modified capsid protein.

32. A transformed cell comprising the polynucleotide of claim 1 or 2, the expression cassette of any one of claims 3-26, and/or the vector of any one of claims 27-31.

33. The transformed cell of claim 32, wherein the polynucleotide, expression cassette, and/or vector is stably incorporated into the cell genome.

34. A transgenic animal comprising the polynucleotide of claim 1 or 2, the expression cassette of any one of claims 3-26, the vector of any one of claims 27-31, and/or the transformed cell of claim 32 or 33.

35. A pharmaceutical composition comprising the polynucleotide of claim 1 or 2, the expression cassette of any one of claims 3-26, the vector of any one of claims 27-31, and/or the transformed cell of claim 32 or 33, and a pharmaceutically acceptable carrier.

36. A recombinant adeno-associated virus (rAAV) vector comprising in 5′ to 3′ direction:
a) a first AAV ITR sequence;
b) a promoter sequence;
c) a transgene nucleic acid molecule, wherein the transgene nucleic acid molecule comprises a nucleic acid sequence encoding an AP4M1 polypeptide;
d) a polyA sequence; and
e) a second AAV ITR sequence.

37. The rAAV vector of claim 36, further comprising an intron sequence.

38. The rAAV vector of claim 36 or 37, wherein the transgene nucleic acid molecule comprises the nucleic acid sequence set forth in SEQ ID NO:5 or SEQ ID NO:40.

39. The rAAV vector of claim 38, wherein the vector comprises SEQ ID NO:1 or SEQ ID NO: 41.

40. The rAAV vector of any one of the preceding claims, wherein the transgene nucleic acid sequence encoding for an AP4M1 polypeptide exhibits at least 5%, at least 10%, at least 20%, at least 30%, at least 50%, at least 75%, at least 100%, at least 200%, at least 300%, at least 500%, or at least 1000% increased expression in a human subject relative to a mutated AP4M1 nucleic acid sequence.

41. The rAAV vector of any one of the preceding claims, wherein the first AAV ITR sequence comprises the nucleic acid sequence set forth in SEQ ID NO:2.

42. The rAAV vector of any one of the preceding claims, wherein the second AAV ITR sequence comprises the nucleic acid sequence set forth in SEQ ID NO:7.

43. The rAAV vector of any one of the preceding claims, wherein the promoter sequence comprises a Rous sarcoma virus (RSV) LTR promoter (optionally with an RSV enhancer), a cytomegalovirus (CMV) promoter, an SV40 promoter, a dihydrofolate reductase promoter, a beta-actin promoter, a phosphoglycerol kinase (PGK) promoter, a U6 promoter, a JetI promoter, an H1 promoter, a CAG promoter, a hybrid chicken beta-actin (CBA) promoter, an MeCP2 promoter, an EF1 promoter, a ubiquitous chicken β-actin hybrid (CBh) promoter, an U1a promoter, an U1b promoter, an MeCP2 promoter, an MeP418 promoter, an MeP426 promoter, a minimal MeCP2 promoter, a VMD2 promoter, an mRho promoter, an EFla promoter, an Ubc promoter, a human β-actin promoter, a synapsin (hSyn) promoter sequence, a TRE promoter, an Ac5 promoter, a Polyhedrin promoter, a CaMKIIa promoter, a Gall promoter, a TEF1 promoter, a GDS promoter, an ADH1 promoter, an Ubi promoter, a Jet promoter, an UsP promoter, or an α-1-antitrypsin (hAAT) promoter.

44. The rAAV vector of any one of the preceding claims, wherein the promoter sequence comprises the nucleic acid sequence set forth in SEQ ID NO:3 or SEQ ID NO:39.

45. The rAAV vector of any one of the preceding claims, wherein the polyA sequence comprises the nucleic acid sequence set forth in SEQ ID NO:6.

46. An rAAV vector of any one of the preceding claims, comprising, in the 5′ to 3′ direction:
a) a first AAV ITR sequence comprising the nucleic acid sequence set forth in SEQ ID NO:2;
b) a promoter sequence comprising the nucleic acid sequence set forth in SEQ ID NO:3;
c) a transgene nucleic acid molecule, wherein the transgene nucleic acid molecule comprises a nucleic acid sequence encoding for an AP4M1 polypeptide, wherein the nucleic acid sequence encoding for an AP4M1 polypeptide comprises the nucleic acid sequence set forth in SEQ ID NO:5;
d) a polyA sequence comprising the nucleic acid sequence set forth in SEQ ID NO:6; and
e) a second AAV ITR sequence comprising the nucleic acid sequence set forth in SEQ ID NO:7.

47. An rAAV vector of any one of the preceding claims, comprising, in the 5′ to 3′ direction:
a) a first AAV ITR sequence comprising the nucleic acid sequence set forth in SEQ ID NO:2;
b) a promoter sequence comprising the nucleic acid sequence set forth in SEQ ID NO:39;
c) a transgene nucleic acid molecule, wherein the transgene nucleic acid molecule comprises a nucleic acid sequence encoding for an AP4M1 polypeptide, wherein the nucleic acid sequence encoding for an AP4M1 polypeptide comprises the nucleic acid sequence set forth in SEQ ID NO:40;
d) a polyA sequence comprising the nucleic acid sequence set forth in SEQ ID NO:6; and
e) a second AAV ITR sequence comprising the nucleic acid sequence set forth in SEQ ID NO:7.

48. An rAAV vector of any one of the preceding claims, comprising, in the 5′ to 3′ direction:
a) a first AAV ITR sequence comprising the nucleic acid sequence set forth in SEQ ID NO:2;
b) a promoter sequence comprising the nucleic acid sequence set forth in SEQ ID NO:3;
c) an intron sequence comprising the nucleic acid sequence set forth in SEQ ID NO:4;
d) a transgene nucleic acid molecule, wherein the transgene nucleic acid molecule comprises a nucleic acid sequence encoding for an AP4M1 polypeptide, wherein the nucleic acid sequence encoding for an AP4M1 polypeptide comprises the nucleic acid sequence set forth in SEQ ID NO:5;
e) a polyA sequence comprising the nucleic acid sequence set forth in SEQ ID NO:6; and
f) a second AAV ITR sequence comprising the nucleic acid sequence set forth in SEQ ID NO:7.

49. An rAAV viral vector comprising:
(i) an AAV capsid protein; and
(ii) an rAAV vector of any one of the preceding claims.

50. The rAAV viral vector of claim 49, wherein the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein.

51. The rAAV viral vector of claim 50, wherein the AAV capsid protein is an AAV9 capsid protein.

52. A pharmaceutical composition comprising:
a) the rAAV viral vector of any one of claims 46-51; and at least one pharmaceutically acceptable excipient and/or additive.

53. The rAAV viral vector of any one of claims 46-51 or the pharmaceutical composition of claim 52 for use in treating a disease and/or disorder involving an AP4M1 gene in a subject in need thereof.

54. The use of claim 53, wherein the disease and/or disorder involving an AP4M1 gene is spastic paraplegia type 50 (SPG50).

55. The use of claim 53 or claim 54, wherein the rAAV viral vector or the pharmaceutical composition is for administration to the subject at a dose ranging from about 10¹¹ to about 10¹⁸ viral vector particles.

56. The use of any of claims 53-55, wherein the rAAV viral vector or the pharmaceutical composition is for administration to the subject at a dose ranging from about 10¹³ to about 10¹⁶ viral vector particles.

57. The use of any one of claims 53-56, wherein the rAAV viral vector or the pharmaceutical composition is for administration to the subject intravenously, intrathecally, intracisterna-magna, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally or intranerve.

58. The use of claim 57, wherein the rAAV viral vector or pharmaceutical composition is for lumbar intrathecal administration.

59. A method of expressing an AP4M1 polynucleotide in a cell, comprising contacting the cell with the polynucleotide of claim 1 or 2, the expression cassette of any one of claims 3-26, the vector of any one of claims 27-31, the rAAV vector of any one of claims 36-51, and/or the pharmaceutical composition of claim 35 or 52, thereby expressing the AP4M1 polynucleotide in the cell.

60. A method of expressing an AP4M1 polynucleotide in a subject, comprising delivering to the subject the polynucleotide of claim 1 or 2, the expression cassette of any one of claims 3-26, the vector of any one of claims 27-31, the rAAV vector of any one of claims 36-51, the pharmaceutical composition of claim 35 or 52, and/or the transformed cell of claim 32 or 33, thereby expressing the AP4M1 polynucleotide in the subject.

61. A method of treating a disorder associated with aberrant expression of an AP4M1 gene or aberrant activity of an AP4M1 gene product in a subject in need thereof, comprising delivering to the subject a therapeutically effective amount of the polynucleotide of claim 1 or 2, the expression cassette of any one of claims 3-26, the vector of any one of claims 27-31, the rAAV vector of any one of claims 36-51, the pharmaceutical composition of claim 35 or 52, and/or the transformed cell of claim 32 or 33, thereby treating the disorder associated with aberrant expression of the AP4M1 gene in the subject.

62. A method of treating spastic paraplegia type 50 (SPG50) in a subject comprising delivering to the subject a therapeutically effective amount of the polynucleotide of claim 1 or 2, the expression cassette of any one of claims 3-26, the vector of any one of claims 27-31, the rAAV vector of any one of claims 36-51, the pharmaceutical composition of claim 35 or 52, and/or the transformed cell of claim 32 or 33, thereby treating SPG50 in the subject.

63. The method of any one of claims 59-62, wherein the polynucleotide,
expression cassette, vector, rAAV vector, pharmaceutical composition, and/or transformed cell is delivered to the nervous system of the subject.

64. The method of claim 63, wherein the polynucleotide, expression cassette, vector, rAAV vector, pharmaceutical composition, and/or transformed cell is delivered by intrathecal, intracerebral, intraventricular, intranasal, intra-aural, intra-ocular, or peri-ocular delivery, or any combination thereof.

65. The method of any one of claims 59-62, wherein the rAAV vector or a pharmaceutical composition thereof is administered to the subject.

66. The method of claim 65, wherein the rAAV viral vector or the pharmaceutical composition thereof is administered to the subject at a dose ranging from about 10¹¹ to about 10¹⁸ viral vector particles.

67. The method of claim 66, wherein the rAAV viral vector or the pharmaceutical composition is administered to the subject at a dose ranging from about 10¹³ to about 10¹⁶ viral vector particles.

68. The method of claim 66, wherein the rAAV viral vector or the pharmaceutical composition is administered to the subject at a dose comprising about 1.25E11, 2.5E11 or 5E11 vector particles.

69. The method of any one of claims 65-68, wherein the rAAV viral vector or the pharmaceutical composition thereof is administered to the subject intravenously, intrathecally, intracisterna-magna, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally or intranerve.

70. The method of claim 69, wherein the rAAV viral vector or the pharmaceutical composition thereof is administered by lumbar intrathecal administration.

71. The method of claim 65, wherein administering the rAAV vector or a pharmaceutical composition thereof increases AP4M1 levels in the cortex, subcortex, brain stem and/or cerebellum of the subject.

72. The method of claim 65, wherein administering the rAAV vector or a pharmaceutical composition thereof induces no immune response or a minimal immune response from the subject.

73. The method of claim 65, wherein administering the rAAV vector or a pharmaceutical composition thereof induces no serum toxicity or a minimal serum toxicity in the subject.

74. The method of claim 65, wherein administering the rAAV vector or a pharmaceutical composition thereof improves motor coordination and/or grip strength in the subject.
